(19) United States
(12) Patent Application Publication
(10) Pub. No.: US 2020/0191697 A1
(43) Pub. Date: Jun. 18, 2020

(54) METHOD FOR EVALUATING THICKNESS AND DENSITY OF ADSORBED METHANE IN PORES CONTRIBUTED BY ORGANIC MATTER, CLAY AND OTHER MINERALS IN MUD SHALE RESERVOIR

(71) Applicant: China University of Petroleum (East China), Qingdao (CN)

(72) Inventors: Fangwen CHEN, Qingdao (CN); Shuangfang LU, Qingdao (CN); Xue DING, Qingdao (CN); Hongqin ZHAO, Qingdao (CN)

(21) Appl. No.: 16/686,129

(22) Filed: Nov. 16, 2019

Related U.S. Application Data
(63) Continuation of application No. PCT/CN2019/087062, filed on May 15, 2019.

(30) Foreign Application Priority Data
Dec. 13, 2018 (CN) 201811521652.4

(51) Int. Cl. G01N 15/08; G01N 33/24

(52) U.S. Cl. CPC G01N 15/0806; G01N 2015/0866; G01N 33/241; G01N 15/088

(57) ABSTRACT

A method for evaluating thickness and density of adsorbed methane in pores contributed by organic matter, clay and other minerals in a mud shale reservoir, including: crushing a sample and selecting three or more subsamples with different meshes to determine TOC, kerogen, whole rock analysis, low-temperature nitrogen adsorption-desorption and methane isotherm adsorption; calculating contents of organic matter in respective subsamples from TOC and kerogen contents; normalizing contents of organic matter, clay and other minerals; evaluating the volume of pores contributed by organic matter, clay and other minerals per unit mass according to contents thereof and low-temperature nitrogen adsorption-desorption; evaluating content of adsorbed methane in organic matter, clay and other minerals per unit mass according to contents thereof and methane isotherm adsorption; and establishing a model for calculating density and thickness of adsorbed methane.

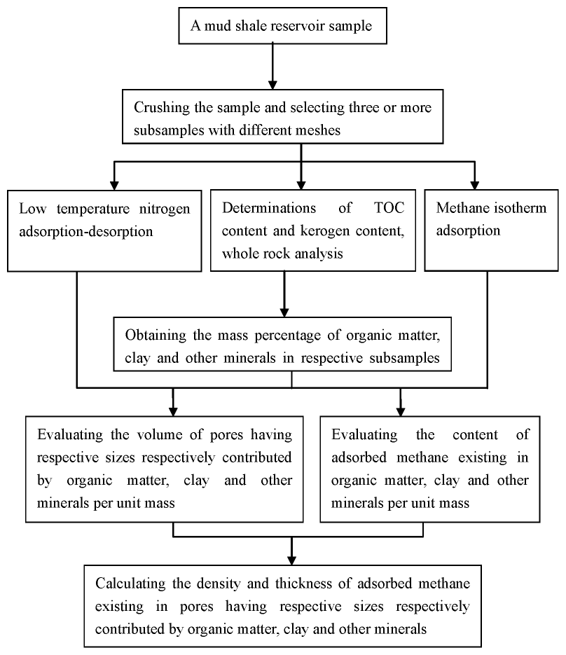

### CROSS-REFERENCE TO RELATED APPLICATIONS

This application is a continuation of International Application No. PCT/CN2019/087062, filed on May 15, 2019, which claims the benefit of priority from Chinese Patent Application No. 201811521652.4, filed on Dec. 13, 2018. The content of the aforementioned applications, including any intervening amendments thereto, are incorporated herein by reference.

### TECHNICAL FIELD

This application relates to natural gas exploration, and more particularly to a method for evaluating thickness and density of adsorbed methane in pores contributed by organic matter, clay and other minerals in a mud shale reservoir.

### BACKGROUND OF THE INVENTION

Shale gas is a natural gas accumulation existing in strata, such as the mudstone and shale capable of generating hydrocarbon, mainly in adsorbed and free states. In a mud shale reservoir, the shale gas exists mainly as free gas and adsorbed gas. The free gas mainly exists in a central space of cracks, macropores and small pores in the mud shale reservoir, while the adsorbed gas is mainly physically adsorbed by an inner surface of nanopores. The primary factors affecting the content of methane adsorbed in the mud shale reservoir includes pore wall composition, pore volume, pore size distribution, temperature, pressure and water content. The specific surface of different kinds of porous media plays a leading role in affecting the amount of adsorbed gas existing in pores. The density of the adsorbed methane is affected not only by temperature but also by pressure. Therefore, when the changing characteristics of thickness and density of adsorbed methane in pores having respective sizes contributed by organic matter, clay and other minerals in the mud shale reservoir over temperature and pressure are determined, the absolute adsorption content of adsorbed methane in the mud shale reservoir can also be accordingly determined, which plays an important role in evaluating shale gas resources, optimizing shale gas exploiting areas and formulating a plan for exploiting a shale gas well.

Currently, it is not appropriate to calculate the absolute adsorption content according to liquid phase density or constant density of methane, because it is difficult to define the boundary between the free gas and the adsorbed gas in pores by experimental methods and to analyze the density of the adsorbed state. Currently, the density of adsorbed shale gas and the thickness of the adsorbed layer are mainly evaluated by molecular simulations, which have the following shortcomings. For example, the solid surface constructed is a flat type rather than an actually circular arc type, that is, the slit-shaped pore space is constructed, in which the superposition effect of the arc-shaped pore wall on the adsorption potential of gas molecules is ignored. The solid structure constructed is too simple to establish a complex organic matter model, affecting the evaluation for the adsorption of methane by organic matter in a mud shale in the molecular simulation. Molecular simulations are limited in system size and molecular number due to computational limitations. When the molecular simulation is used to predict the adsorption, the density is calculated through a function of fugacity rather than pressure, where the fugacity is broadly defined as the deviation in the vapor pressure between a real gas and the corresponding ideal gas. There is a lack of experimental data to support the results of molecular simulations or there are large errors in the comparison with the experimental analysis results. For example, there is a lack of experimental support for the density and thickness of the adsorbed gas, and there is a big difference in the amount of the adsorbed gas existing in per unit mass of an absorbent between the molecular simulations and experiments.

Therefore, according to experimental results of organic carbon content, kerogen content, whole rock analysis, low temperature nitrogen adsorption-desorption and isothermal adsorption of methane, a first model is established herein for evaluating contribution of organic matter, clay and other minerals to the volume of pores having respective sizes to determine contribution of organic matter, clay and other minerals per unit mass to the volume of pores having respective sizes. Then a second model is established for evaluating content of adsorbed methane existing in organic matter, clay and other minerals to determine content of adsorbed methane existing in organic matter, clay and other minerals per unit mass. Finally a third model is established for evaluating thickness and density of adsorbed methane in pores having respective sizes contributed by organic matter, clay and other minerals to quantitatively determine the change of thickness and density of adsorbed methane in pores having respective sizes contributed by organic matter, clay and other minerals over temperature and pressure.

### SUMMARY OF THE INVENTION

An object of the invention is to provide a method for evaluating thickness and density of adsorbed methane in pores contributed by organic matter, clay and other minerals in a mud shale reservoir to overcome the defects in the prior art that the thickness and density of adsorbed methane in pores having respective sizes contributed by organic matter, clay and other minerals in the mud shale reservoir cannot be effectively evaluated, achieving the quantitative evaluation of thickness and density of adsorbed methane in pores contributed by organic matter, clay and other minerals in the mud shale reservoir.

Technical solutions of the invention are described as follows.

The invention provides a method for evaluating thickness and density of adsorbed methane in pores contributed by organic matter, clay and other minerals in a mud shale reservoir, comprising:

1) crushing a mud shale reservoir sample to produce a plurality of subsamples; and selecting three or more subsamples varying in mesh for determinations of organic carbon content and kerogen content, whole rock analysis, and determinations of low temperature nitrogen adsorption-desorption and methane isotherm adsorption;

where mass percentages of organic carbon in respective subsamples are w_{TOC-1}⁰, w_{TOC-2}⁰, . . . and w_{TOC-n}⁰ (%), respectively; mass percentages of carbon in kerogen in respective subsamples are w_{C-1}, w_{C-2}, . . . and w_{C-n} (%), respectively; mass percentages of clay in respective subsamples are w_{clay-1}⁰, w_{clay-2}⁰, . . . and w_{clay-n}⁰ (%), respectively; and mass percentages of other minerals in respective subsamples are wₒₜₕₑᵣₛ₋⁰, wₒₜₕₑᵣₛ₋₂⁰, . . . and wₒₜₕₑᵣₛ₋ₙ⁰ (%), respectively; pores in respective subsamples per unit mass having a size respectively of <2 nm, 2-5 nm, 5-10 nm, 10-20 nm, 20-50 nm, 50-100 nm and 100-200 nm have a volume of Vᵢⱼ (cm³/g); respective subsamples per unit mass have an adsorbed methane content of Q_{ixy} (m³/t) under a temperature of Tₓ and a pressure of P_{y},

where

i is a number of respective subsamples of the mud shale reservoir, and is selected from 1, 2, 3, . . . , n; j is a number of pore sizes from small to large, and is selected from 1, 2, . . . , 7; x is a number of temperature from low to high, and is selected from 1, 2, . . . , m; y is a number of pressure from low to high, and is selected from 1, 2, . . . , z;

2) substituting the mass percentages of organic carbon (w_{TOC-1}⁰, w_{TOC-2}⁰, . . . and w_{TOC-n}⁰ and the corresponding mass percentages of carbon in kerogen (w_{C-1}, w_{C-2}, . . . and w_{C-n}) in respective subsamples into the following equation to obtain mass percentages of organic matter in respective subsamples (w_{TOM-1}⁰, w_{TOM-2}⁰, . . . and w_{TOM-n}⁰;

w_{TOM-i}⁰=w_{TOC-i}⁰/w_{C-i}×100%;

where w_{TOM-i}⁰(%) is an unnormalized mass percentage of organic matter in respective subsamples; w_{TOC-i}⁰ (%) is an experimentally measured mass percentage of organic carbon in respective subsamples; w_{C-i} (%) is an experimentally measured mass percentage of carbon in kerogen in respective subsamples; i is the number of respective subsamples of the mud shale reservoir, and is selected from 1, 2, 3, . . . , n; and

normalizing the mass percentages of organic matter, clay and other minerals in respective subsamples according to the following equations; wherein a sum of the mass percentages of organic matter, clay and other minerals in respective subsamples is 100%; the normalized mass percentages of organic matter, clay and other minerals is in respective subsamples are respectively w_{TOM-i} (%), w_{clay-i} (%) and wₒₜₕₑᵣₛ₋ᵢ (%);

w_{TOM-i}=w_{TOM-i}⁰×100%

w_{clay-i}=w_{clay-i}⁰×(100−w_{TOM-i}⁰)/100%

wₒₜₕₑᵣₛ₋ᵢ=wₒₜₕₑᵣₛ₋ᵢ⁰×(100−w_{TOM-i}⁰)/100%

where w_{TOM-i}⁰, w_{clay-i}⁰and wₒₜₕₑᵣₛ₋ᵢ⁰ are mass percentages of organic matter, clay and other minerals in respective subsamples before normalization, respectively; i is the number of respective subsamples of the mud shale reservoir, and is selected from 1, 2, 3, . . . , n;

3) establishing a first equation set and a first target function according to the normalized mass percentages of organic matter (w_{TOM-1}, w_{TOM-}, . . . and w_{TOM-n}), the normalized mass percentages of clay (w_{clay-1}, w_{clay-2}, . . . and w_{clay-n}), and the normalized mass percentage of other minerals (wₒₜₕₑᵣ₋₁, wₒₜₕₑᵣ₋₂, . . . and wₒₜₕₑᵣ₋ₙ) in respective subsamples obtained in step (2) and the volume Vᵢᵢ (cm³/g) of pores having a size respectively of <2 nm, 2-5 nm, 5-10 nm, 10-20 nm, 20-50 nm, 50-100 nm and 100-200 nm in respective subsamples per unit mass obtained in step (1);

where in the case of a minimum value of the first target function f(V_{TOM-j}, V_{clay-j}, Vₒₜₕₑᵣ₋ⱼ), a volume of pores having a size numbered as j contributed by organic matter per unit mass is V_{TOM-j} (cm³/g) a volume of pores having a size numbered as j contributed by clay per unit mass is V_{clay-j} (cm³/g), and a volume of pores having a size numbered as j contributed by other minerals per unit mass is Vₒₜₕₑᵣₛ₋ⱼ (cm³/g);

$${w}_{TOM \text{-} 1} \times {V}_{TOM \text{-} j} + {w}_{clay \text{-} 1} \times {V}_{clay \text{-} j} + {w}_{others \text{-} 1} \times {V}_{others \text{-} j} = {V}_{1 j}$$

$${w}_{TOM \text{-} 2} \times {V}_{TOM \text{-} j} + {w}_{clay \text{-} 2} \times {V}_{clay \text{-} j} + {w}_{others \text{-} 2} \times {V}_{others \text{-} j} = {V}_{2 j}$$

$${w}_{TOM \text{-} 3} \times {V}_{TOM \text{-} j} + {w}_{clay \text{-} 3} \times {V}_{clay \text{-} j} + {w}_{others \text{-} 3} \times {V}_{others \text{-} j} = {V}_{3 j}$$

$$\begin{matrix}⋯ & ⋯ & ⋯\end{matrix}$$

$${w}_{TOM \text{-} n} \times {V}_{TOM \text{-} j} + {w}_{clay \text{-} n} \times {V}_{clay \text{-} j} + {w}_{others \text{-} n} \times {V}_{others \text{-} j} = {V}_{nj}$$

$${V}_{TOM \text{-} j} > 0 , {V}_{clay \text{-} j} > 0 , {V}_{others \text{-} j} > 0$$

$$f ( {V}_{TOM \text{-} j} , {V}_{clay \text{-} j} , {V}_{others \text{-} j} ) = {\sum }_{i = 1}^{n} {( {V}_{ij} - {w}_{TOM \text{-} i} \times {V}_{TOM \text{-} j} - {w}_{clay \text{-} i} \times {V}_{clay \text{-} j} - {w}_{others \text{-} i} \times {V}_{others \text{-} j} )}^{2} ;$$

where V_{TOM-j}, V_{clay-j} and Vₒₜₕₑᵣₛ₋ⱼ are the volumes of pores having a size numbered as j respectively contributed by organic matter, clay and other minerals per unit mass; j is a number of pore size from small to large, and is selected from 1, 2, . . . 6 and 7; i is the number of respective subsamples of the mud shale reservoir, and is selected from 1, 2, 3, . . . , n;

4) establishing a second equation set and a second target function using the normalized mass percentage of organic matter (w_{TOM-1}, w_{TOM-2}, . . . and w_{TOM-n}), the normalized mass percentage of clay (w_{clay-1}, w_{clay-2}, . . . and w_{clay-n}), and the normalized mass percentage of other minerals (wₒₜₕₑᵣₛ₋₁, wₒₜₕₑᵣₛ₋₂, . . . and wₒₜₕₑᵣₛ₋ₙ) in respective subsamples in step (2) and the content Q_{ixy} of adsorbed methane existing in respective subsamples per unit mass obtained in step (1) under a temperature of Tₓ and a pressure of P_{y};

where in the case of a minimum value of the second target function f(Q_{TOM-xy}, Q_{clay-xy}, Q_{others-xy}), a temperature of Tₓ and a pressure of P_{y}, a content of adsorbed methane existing in organic matter per unit mass is Q_{TOM-xy}, a content of adsorbed methane existing in clay per unit mass is Q_{clay-xy}, and a content of adsorbed methane existing in other minerals per unit mass is Q_{others-xy},

$${w}_{TOM \text{-} 1} \times {Q}_{TOM \text{-} xy} + {w}_{clay \text{-} 1} \times {Q}_{clay \text{-} xy} + {w}_{others \text{-} 1} \times {Q}_{others \text{-} xy} = {Q}_{1 xy}$$

$${w}_{TOM \text{-} 2} \times {Q}_{TOM \text{-} xy} + {w}_{clay \text{-} 2} \times {Q}_{clay \text{-} xy} + {w}_{others \text{-} 2} \times {Q}_{others \text{-} xy} = {Q}_{2 xy}$$

$${w}_{TOM \text{-} 3} \times {Q}_{TOM \text{-} xy} + {w}_{clay \text{-} 3} \times {Q}_{clay \text{-} xy} + {w}_{others \text{-} 3} \times {Q}_{others \text{-} xy} = {Q}_{3 xy}$$

$$\begin{matrix}⋯ & ⋯ & ⋯\end{matrix}$$

$${w}_{TOM \text{-} n} \times {Q}_{TOM \text{-} xy} + {w}_{clay \text{-} n} \times {Q}_{clay \text{-} xy} + {w}_{others \text{-} n} \times {Q}_{others \text{-} xy} = {Q}_{nxy}$$

$${Q}_{TOM \text{-} xy} > 0 , {Q}_{clay \text{-} xy} > 0 , {Q}_{others \text{-} xy} > 0$$

$$f ( {Q}_{TOM \text{-} xy} , {Q}_{clay \text{-} xy} , {Q}_{others \text{-} xy} ) = {\sum }_{i = 1}^{n} {( {Q}_{ixy} - {w}_{TOM \text{-} i} \times {Q}_{TOM \text{-} xy} - {w}_{clay \text{-} i} \times {Q}_{clay \text{-} xy} - {w}_{others \text{-} i} \times {Q}_{others \text{-} xy} )}^{2}$$

where, Q_{TOM-xy} (m³/t), Q_{clay-xy} (m³/t) and Q_{others-xy} (m³/t) are the contents of adsorbed methane respectively existing in organic matter, clay and other minerals per unit mass under a temperature of Tₓ (° C.) and a pressure P_{y} (MPa);

Q_{ixy} (m³/t) is a content of adsorbed methane existing in subsample i per unit mass under a temperature of Tₓ and a pressure of P_{y}; wherein i is the number of respective subsamples of the mud shale reservoir, and is selected from 1, 2, 3, . . . , n; x is the number of temperature from low to high, and is selected from 1, 2, . . . , m; and y is the number of pressure from low to high, and is selected from 1, 2, . . . , z;

5) calculating V_{absorbed by TOM-jxy} according to the following equations based on step (3) by approximating the pores contributed by organic matter per unit mass to cylinders with corresponding pore size;

where V_{absorbed by TOM-jxy} is the volume of pores occupied by adsorbed methane in pores numbered j contributed by organic matter per unit mass under a temperature of Tₓ and a pressure of P_{y}; when the pores j contributed by organic matter per unit mass have a size D_{TOM-j} lower than 0.38 nm, V_{absorbed by TOM-jxy}=0; when the D_{TOM-j} is not more than twice a thickness h_{absorbed by TOM-jxy} of adsorbed methane and is not less than 0.38 nm, V_{absorbed by TOM-jxy}=V_{TOM-j}; and when the D_{TOM-j} is more than twice the thickness h_{absorbed by TOM-jxy} of adsorbed methane and is not less than 0.38 nm,

$${V}_{absorbed by TOM \text{-} jxy} = \frac{4 {D}_{TOM \text{-} j} \times {h}_{absorbed by TOM \text{-} jxy} - 4 {h}_{absorbed by TOM \text{-} jxy}^{2}}{{D}_{TOM \text{-} j}^{2}} \times {V}_{TOM \text{-} j} ;$$

$${V}_{absorbed by TOM \text{-} jxy} = \{ \begin{matrix}\begin{matrix}0 & ( {D}_{TOM \text{-} j} < 0.38 nm )\end{matrix} \\ \begin{matrix}{V}_{TOM \text{-} j} & ( 2 {h}_{absorbed by TOM \text{-} jxy} \geq {D}_{TOM \text{-} j} , {D}_{TOM \text{-} j} \geq 0.38 nm )\end{matrix} \\ \frac{4 {D}_{TOM \text{-} j} \times {h}_{absorbed by TOM \text{-} jxy} - 4 {h}_{absorbed by TOM \text{-} jxy}^{2}}{{D}_{TOM \text{-} j}^{2}} \times {V}_{TOM \text{-} j} \\ ( 2 {h}_{absorbed by TOM \text{-} jxy} < {D}_{TOM \text{-} j} , {D}_{TOM \text{-} j} \geq 0.38 nm ) ;\end{matrix}$$

where, V_{absorbed by TOM-jxy} (cm³/g) is the volume of pores occupied by adsorbed methane in pores numbered j contributed by organic matter per unit mass under a temperature of Tₓ and a pressure of P_{y}; V_{TOM-j} (cm³/g) is the volume of pores numbered j contributed by organic matter per unit mass; D_{TOM-j} (nm) is the size of pores numbered j contributed by organic matter; h_{absorbed by TOM-jxy} (nm) is the thickness of adsorbed methane in pores numbered j contributed by organic matter; j is the number of pore sizes from small to large, and is selected from 1, 2, . . . , 7; x is the number of temperature from low to high, and is selected from 1, 2, . . . , m; and y is the number of pressure from low to high, and is selected from 1, 2, . . . , z;

calculating V_{absorbed by clay-jxy} according to the following equations based on step (3) by approximating the pores contributed by clay per unit mass to cylinders with corresponding pore size;

where V_{absorbed by clay-jxy} is the volume of pores occupied by adsorbed methane in pores numbered j contributed by organic matter per unit mass under a temperature of Tₓ and a pressure of P_{y}; when the pores j contributed by organic matter per unit mass have a size D_{clay-j} lower than 0.38 nm, V_{absorbed by clay-jxy}=0; when the D_{clay-j} is not more than twice a thickness h_{absorbed by clay-jxy} of adsorbed methane and is not less than 0.38 nm, V_{absorbed by clay-jxy}=V_{clay-j}; and when the D_{clay-j} is more than twice the thickness h_{absorbed by clay-jxy} of adsorbed methane and is not less than 0.38 nm,

$${V}_{absorbed by clay \text{-} jxy} = \frac{4 {D}_{clay \text{-} j} \times {h}_{absorbed by clay \text{-} jxy} - 4 {h}_{absorbed by clay \text{-} jxy}^{2}}{{D}_{clay \text{-} j}^{2}} \times {V}_{clay \text{-} j} ;$$

$${V}_{absorbed by clay \text{-} jxy} = \{ \begin{matrix}\begin{matrix}0 & ( {D}_{clay \text{-} j} < 0.38 nm )\end{matrix} \\ \begin{matrix}{V}_{clay \text{-} j} & ( 2 {h}_{absorbed by clay \text{-} jxy} \geq {D}_{clay \text{-} j} , {D}_{clay \text{-} j} \geq 0.38 nm )\end{matrix} \\ \frac{4 {D}_{clay \text{-} j} \times {h}_{absorbed by clay \text{-} jxy} - 4 {h}_{absorbed by clay \text{-} jxy}^{2}}{{D}_{clay \text{-} j}^{2}} \times {V}_{clay \text{-} j} \\ ( 2 {h}_{absorbed by clay \text{-} jxy} < {D}_{clay \text{-} j} , {D}_{clay \text{-} j} \geq 0.38 nm )\end{matrix}$$

where, V_{absorbed by clay-jxy (cm}³/g) is the volume of pores occupied by adsorbed methane in pores numbered j contributed by clay per unit mass under a temperature of Tₓ and a pressure of P_{y}; V_{clay-j} (cm³/g) is the volume of pores numbered j contributed by clay per unit mass; D_{clay-j} (nm) is the size of pores numbered j contributed by clay per unit mass; h_{absorbed by clay-jxy} (nm) is the thickness of adsorbed methane in pores numbered j contributed by clay; j is the number of pore sizes from small to large, and is selected from 1, 2, . . . , 7; x is the number of temperature from low to high, and is selected from 1, 2, . . . , m; and y is the number of pressure from low to high, and is selected from 1, 2, . . . , z;

calculating V_{absorbed by others-jxy} according to the following equations based on step (3) by approximating the pores contributed by other minerals per unit mass to cylinders with corresponding pore size;

where, V_{absorbed by others-jxy} is the volume of pores occupied by adsorbed methane in pores numbered j contributed by organic matter per unit mass under a temperature of Tₓ and a pressure of P_{y}; when the pores j contributed by organic matter per unit mass have a size Dₒₜₕₑᵣₛ₋ⱼ lower than 0.38 nm, V_{absorbed by others-jxy}=0; when the Dₒₜₕₑᵣₛ₋ⱼ is not more than twice a thickness h_{absorbed by others-jxy} of adsorbed methane and is not less than 0.38 nm, V_{absorbed by others-jxy}=Vₒₜₕₑᵣ₋ⱼ; and when the Dₒₜₕₑᵣ₋ⱼ is more than twice the thickness h_{absorbed by others-jxy} of adsorbed methane and is not less than 0.38 nm,

$${V}_{absorbed by others \text{-} jxy} = \frac{4 {D}_{others \text{-} j} \times {h}_{absorbed by others \text{-} jxy} - 4 {h}_{absorbed by others \text{-} jxy}^{2}}{{D}_{others \text{-} j}^{2}} \times {V}_{others \text{-} j} ;$$

$${V}_{absorbed by others \text{-} jxy} = \{ \begin{matrix}\begin{matrix}0 & ( {D}_{others \text{-} j} < 0.38 nm )\end{matrix} \\ \begin{matrix}{V}_{others \text{-} j} & ( 2 {h}_{absorbed by others \text{-} jxy} \geq {D}_{others \text{-} j} , {D}_{others \text{-} j} \geq 0.38 nm )\end{matrix} \\ \frac{4 {D}_{others \text{-} j} \times {h}_{absorbed by others \text{-} jxy} - 4 {h}_{absorbed by others \text{-} jxy}^{2}}{{D}_{others \text{-} j}^{2}} \times {V}_{others \text{-} j} \\ ( 2 {h}_{absorbed by others \text{-} jxy} < {D}_{others \text{-} j} , {D}_{others \text{-} j} \geq 0.38 nm )\end{matrix}$$

where, V_{absorbed by others-jxy} (cm³/g) is the volume of pores occupied by adsorbed methane existing in pores numbered j contributed by other minerals per unit mass under a temperature of Tₓ and a pressure of P_{y}; Vₒₜₕₑᵣₛ₋ⱼ (cm³/g) is the volume of pores numbered j contributed by other minerals per unit mass; Dₒₜₕₑᵣₛ₋ⱼ (nm) is the size of pores numbered j contributed by other minerals per unit mass; h_{absorbed by others-jxy} (nm) is the thickness of adsorbed methane in pores numbered j contributed by other minerals; j is the number of pore sizes from small to large, and is selected from 1, 2, . . . , 7; x is the number of temperature from low to high, and is selected from 1, 2, . . . , m; and y is the number of pressure from low to high, and is selected from 1, 2, . . . , z;

6) establishing a third equation set and a third target function based on steps (4) and (5) according to the facts that a density of adsorbed methane is lower than that of solid methane but greater than that of free methane; the density of adsorbed methane in pores of organic matter decreases with the increase of pore size; and the density of adsorbed methane decreases with the increase of temperature while increases with the increase of pressure;

where in the case of a minimum value of the third target function f(ρ_{absorbed by TOM-jxy}, h_{absorbed by TOM-jxy}), a density ρ_{absorbed by TOM-jxy} and a thickness h_{absorbed by TOM-jxy} of adsorbed methane in pores numbered j contributed by organic matter per unit mass under a temperature of Tₓ and a pressure of P_{y} can be obtained;

$$\frac{22.4}{M} {\sum }_{j = 1}^{7} [ {V}_{absorbed by TOM \text{-} jxy} \times ( {ρ}_{absorbed by TOM \text{-} jxy} - {ρ}_{free \text{-} xy} ) ] = {Q}_{TOM \text{-} xy}$$

$${ρ}_{solid} > {ρ}_{absorbed by TOM \text{-} 1 xy} > {ρ}_{absorbed by TOM \text{-} 2 xy} > … > {ρ}_{absorbed by TOM \text{-} 6 xy} > {ρ}_{absorbed by TOM \text{-} 7 jxy} > {ρ}_{free \text{-} xy}$$

$${ρ}_{solid} > {ρ}_{absorbed by TOM \text{-} j 1 y} > {ρ}_{absorbed by TOM \text{-} j 2 y} > … > {ρ}_{absorbed by TOM \text{-} j ( m \text{-} 1 ) y} > {ρ}_{absorbed by TOM \text{-} jmy} > {ρ}_{free \text{-} my}$$

$${ρ}_{solid} > {ρ}_{absorbed by TOM \text{-} jxz} > {ρ}_{absorbed by TOM \text{-} jx ( z \text{-} 1 )} > … > {ρ}_{absorbed by TOM \text{-} jx 2} > {ρ}_{absorbed by TOM \text{-} jx 1} > {ρ}_{free \text{-} x 1}$$

$${h}_{absorbed by TOM \text{-} 1 xy} > {h}_{absorbed by TOM \text{-} 2 xy} > … > {h}_{absorbed by TOM \text{-} 6 xy} > {h}_{absorbed by TOM \text{-} 7 xy}$$

$${h}_{absorbed by TOM \text{-} j 1 y} > {h}_{absorbed by TOM \text{-} j 2 y} > … > {h}_{absorbed by TOM \text{-} j ( m \text{-} 1 ) y} > {h}_{absorbed by TOM \text{-} jmy}$$

$${h}_{absorbed by TOM \text{-} jxz} > {h}_{absorbed by TOM \text{-} jx ( z \text{-} 1 )} > … > {h}_{absorbed by TOM \text{-} jx 2} > {h}_{absorbed by TOM \text{-} jx 1}$$

$$f ( {ρ}_{absorbed by TOM \text{-} jxy} , {h}_{absorbed by TOM \text{-} jxy} ) = {\sum }_{x = 1}^{m} {\sum }_{y = 1}^{z} ( {Q}_{TOM \text{-} xy} - {( \frac{22.4}{M} {\sum }_{j = 1}^{7} [ {V}_{absorbed by TOM \text{-} jxy} \times ( {ρ}_{absorbed by TOM \text{-} jxy} - {ρ}_{free \text{-} xy} ) ] )}^{2}$$

where, V_{absorbed by TOM-jxy} (cm³/g) is the volume of adsorbed methane in pores numbered j contributed by organic matter per unit mass under a temperature of Tₓ and a pressure of P_{y}; ρ_{absorbed by TOM-jxy} (kg/m³) is a density of adsorbed methane in pores numbered j contributed by organic matter per unit mass under a temperature of Tₓ and a pressure of P_{y}; ρ_{free-xy} (kg/m³) is a density of free methane under a temperature of Tₓ and a pressure of P_{y}; Q_{TOM-xy} (m³/t) is the content of adsorbed methane existing in organic matter per unit mass; ρ_{solid} (kg/m³) is a density of solid methane; h_{absorbed by TOM-jxy} (nm) is the thickness of adsorbed methane in pores numbered j contributed by organic matter; M is the molar mass of methane referring to 16.0425 g/mol; j is the number of pore sizes from small to large, and is selected from 1, 2, . . . , 7; x is the number of temperature from low to high, and is selected from 1, 2, . . . , m; and y is the number of pressure from low to high, and is selected from 1, 2, . . . , z;

establishing a forth equation set and a forth target function based on steps (4) and (5) according to the facts that the density of adsorbed methane is lower than that of solid methane but greater than that of free methane, the density of adsorbed methane in the pores of clay decreases with the increase of pore size, and the density of adsorbed methane decreases with the increase of temperature while increases with the increase of pressure;

where in the case of a minimum value of the forth target function f(ρ_{absorbed by clay jxy}, h_{absorbed by clay-jxy}), a density ρ_{absorbed by clay-jxy} and a thickness h_{absorbed by clay-jxy} of adsorbed methane in pores numbered j contributed by clay per unit mass under a temperature of Tₓ and a pressure of P_{y} can be obtained;

$$\frac{22.4}{M} {\sum }_{j = 1}^{7} [ {V}_{absorbed by clay \text{-} jxy} \times ( {ρ}_{absorbed by clay \text{-} jxy} - {ρ}_{free \text{-} xy} ) ] = {Q}_{clay \text{-} xy}$$

$${ρ}_{solid} > {ρ}_{absorbed by clay \text{-} 1 xy} > {ρ}_{absorbed by clay \text{-} 2 xy} > … > {ρ}_{absorbed by clay \text{-} 6 xy} > {ρ}_{absorbed by clay \text{-} 7 jxy} > {ρ}_{freexy}$$

$${ρ}_{solid} > {ρ}_{absorbed by clay \text{-} j 1 y} > {ρ}_{absorbed by clay \text{-} j 2 y} > … > {ρ}_{absorbed by clay \text{-} j ( m \text{-} 1 ) y} > {ρ}_{absorbed by clay \text{-} jmy} > {ρ}_{free \text{-} my}$$

$${ρ}_{solid} > {ρ}_{absorbed by clay \text{-} jxz} > {ρ}_{absorbed by clay \text{-} jx ( z \text{-} 1 )} > … > {ρ}_{absorbed by clay \text{-} jx 2} > {ρ}_{absorbed by clay \text{-} jx 1} > {ρ}_{free \text{-} x 1}$$

$${h}_{absorbed by clay \text{-} 1 xy} > {h}_{absorbed by clay \text{-} 2 xy} > … > {h}_{absorbed by clay \text{-} 6 xy} > {h}_{absorbed by clay \text{-} 7 xy}$$

$${h}_{absorbed by clay \text{-} j 1 y} > {h}_{absorbed by clay \text{-} j 2 y} > … > {h}_{absorbed by clay \text{-} j ( m \text{-} 1 ) y} > {h}_{absorbed by clay \text{-} jmy}$$

$${h}_{absorbed by clay \text{-} jxz} > {h}_{absorbed by clay \text{-} jx ( z \text{-} 1 )} > … > {h}_{absorbed by clay \text{-} jx 2} > {h}_{absorbed by clay \text{-} jx 1}$$

$$f ( {ρ}_{absorbed by clay \text{-} jxy} , {h}_{absorbed by clay \text{-} jxy} ) = {\sum }_{x = 1}^{m} {\sum }_{y = 1}^{z} ( {Q}_{clay \text{-} xy} - {( \frac{22.4}{M} {\sum }_{j = 1}^{7} [ {V}_{absorbed by clay \text{-} jxy} \times ( {ρ}_{absorbed by clay \text{-} jxy} - {ρ}_{freexy} ) ] )}^{2}$$

where, V_{absorbed by clay-jxy} (cm³/g) is the volume of adsorbed methane in pores numbered j contributed by clay per unit mass under a temperature of Tₓ and a pressure of P_{y}; ρ_{absorbed by clay-xy} (kg/m³) is a density of adsorbed methane in pores numbered j contributed by clay per unit mass under a temperature of Tₓ and a pressure of P_{y}; ρ_{free-xy} (kg/m³) is the density of free methane under a temperature of Tₓ and a pressure of P_{y}; Q_{clay-xy} (m³/t) is the content of adsorbed methane existing in clay per unit mass; ρ_{solid} (kg/m³) is the density of solid methane; h_{absorbed by clay-jxy} (nm) is the thickness of adsorbed methane in pores numbered j contributed by clay; M is the molar mass of methane referring to 16.0425 g/mol; j is the number of pore sizes from small to large, and is selected from 1, 2, . . . , 7; x is the number of temperature from low to high, and is selected from 1, 2, . . . , m; y is the number of pressure from low to high, and is selected from 1, 2, . . . , z;

establishing a fifth equation set and a fifth target function based on steps (4) and (5) according to the facts that the density of adsorbed methane is lower than that of solid methane but greater than that of free methane, the density of adsorbed methane in the pores of other minerals decreases with the increase of pore size, and the density of adsorbed methane decreases with the increase of temperature while increases with the increase of pressure;

where in the case of a minimum value of the fifth target function f(ρ_{absorbed by others-jxy}, h_{absorbed by others-jxy}), a density ρ_{absorbed by others-jxy} and a thickness h_{absorbed by others-jxy} of adsorbed methane in pores numbered j contributed by other minerals per unit mass under a temperature of Tₓ and a pressure of P_{y} can be obtained;

$$\frac{22.4}{M} {\sum }_{j = 1}^{7} [ {V}_{absorbed by others \text{-} jxy} \times ( {ρ}_{absorbed by others \text{-} jxy} - {ρ}_{free \text{-} xy} ) ] = {Q}_{others \text{-} xy}$$

$${ρ}_{solid} > {ρ}_{absorbed by others \text{-} 1 xy} > {ρ}_{absorbed by others \text{-} 2 xy} > … > {ρ}_{absorbed by others \text{-} 6 xy} > {ρ}_{absorbed by others \text{-} 7 jxy} > {ρ}_{free \text{-} xy}$$

$${ρ}_{solid} > {ρ}_{absorbed by others \text{-} j 1 y} > {ρ}_{absorbed by others \text{-} j 2 y} > … > {ρ}_{absorbed by others \text{-} j ( m \text{-} 1 ) y} > {ρ}_{absorbed by others \text{-} jmy} > {ρ}_{free \text{-} my}$$

$${ρ}_{solid} > {ρ}_{absorbed by others \text{-} jxz} > {ρ}_{absorbed by others \text{-} jx ( z \text{-} 1 )} > … > {ρ}_{absorbed by others \text{-} jx 2} > {ρ}_{absorbed by others \text{-} jx 1} > {ρ}_{free \text{-} x 1}$$

$${h}_{absorbed by others \text{-} 1 xy} > {h}_{absorbed by others \text{-} 2 xy} > … > {h}_{absorbed by others \text{-} 6 xy} > {h}_{absorbed by others \text{-} 7 xy}$$

$${h}_{absorbed by others \text{-} j 1 y} > {h}_{absorbed by others \text{-} j 2 y} > … > {h}_{absorbed by others \text{-} j ( m \text{-} 1 ) y} > {h}_{absorbed by others \text{-} jmy}$$

$${h}_{absorbed by others \text{-} jxz} > {h}_{absorbed by others \text{-} jx ( z \text{-} 1 )} > … > {h}_{absorbed by others \text{-} jx 2} > {h}_{absorbed by others \text{-} jx 1}$$

$$f ( {ρ}_{absorbed by others \text{-} jxy} , {h}_{absorbed by others \text{-} jxy} ) = {\sum }_{x = 1}^{m} {\sum }_{y = 1}^{z} ( {Q}_{others \text{-} xy} - {( \frac{22.4}{M} {\sum }_{j = 1}^{7} [ {V}_{absorbed by others \text{-} jxy} \times ( {ρ}_{absorbed by others \text{-} jxy} - {ρ}_{free \text{-} xy} ) ] )}^{2}$$

where, V_{absorbed by others-jxy} (cm³/g) is the volume of adsorbed methane in pores numbered j contributed by other minerals per unit mass under a temperature of Tₓ and a pressure of P_{y}; ρ_{absorbed by others-jxy} (kg/m³) is a density of adsorbed methane in pores numbered j contributed by other minerals per unit mass under a temperature of Tₓ and a pressure of P_{y}; ρ_{free-xy} (kg/m³) is the density of free methane under a temperature of Tₓ and a pressure of P_{y}; Q_{others-xy} (m³/t) is the content of adsorbed methane existing in other minerals per unit mass; ρ_{solid} (kg/m³) is the density of solid methane; h_{absorbed by others-jxy} (nm) is the thickness of adsorbed methane in pores numbered j contributed by other minerals; M is the molar mass of methane referring to 16.0425 g/mol; j is the number of pore sizes from small to large, and is selected from 1, 2, . . . , 7; x is the number of temperature from low to high, and is selected from 1, 2, . . . , m; y is the number of pressure from low to high, and is selected from 1, 2, . . . , z.

### BRIEF DESCRIPTION OF THE DRAWINGS

The FIGURE is a flow chart showing the method of the invention for evaluating thickness and density of adsorbed methane in pores contributed by organic matter, clay and other minerals in a mud shale reservoir.

### DETAILED DESCRIPTION OF EMBODIMENTS

### Example 1

As shown in the FIGURE, the invention provided a method for evaluating thickness and density of adsorbed methane in pores contributed by organic matter, clay and other minerals in a mud shale reservoir, which was described as follows.

1) A mud shale reservoir sample was crushed into a plurality of subsamples, of which 5 subsamples respectively of 20-40 mesh, 40-60 mesh, 60-80 mesh, 80-100 mesh and 100-120 mesh were selected for determinations of TOC content and kerogen content, whole rock analysis, and analysis of low temperature nitrogen adsorption-desorption and methane isotherm adsorption. The obtained mass percentages of TOC in respective subsamples were 1.28%, 1.10%, 2.07%, 2.22% and 2.94%, respectively; the obtained mass percentages of carbon in kerogen were 86.12%, 86.72%, 87.01%, 85.57% and 87.98%, respectively; the obtained mass percentages of clay were 41.6%, 42.2%, 23.0%, 25.7% and 30.3%, respectively; and the obtained mass percentages of other minerals were 58.4%, 57.8%, 77.0%, 74.3% and 69.7%, respectively. The obtained volume Vᵢⱼ (cm³/g) of pores in respective subsamples per unit mass having a size respectively of <2 nm, 2-5 nm, 5-10 nm, 10-20 nm, 20-50 nm, 50-100 nm and 100-200 nm in the low temperature nitrogen adsorption-desorption was shown in Table 1. After the methane isotherm adsorption, the obtained adsorbed methane content of Q_{ixy} (m³/t) in respective subsamples per unit mass under 30° C. and 1 MPa, 2 MPa, 3 MPa, 4 MPa, 5 MPa, 6 MPa, 7 MPa, 8 MPa, 9 MPa and 10 MPa was shown in Table 2.

TABLE 1
Volume of Pores with Different Sizes in Respective Subsamples
Per Unit Mass Having (×10⁻³ cm³/g)

| Subsample | Size | | | | | | |
|---|---|---|---|---|---|---|---|
| No. | <2 nm | 2-5 nm | 5-10 nm | 10-20 nm | 20-50 nm | 50-100 nm | 100-200 nm |
| 1 | 0.29 | 1.16 | 0.84 | 1.09 | 0.74 | 0.44 | 0.23 |
| 2 | 0.34 | 1.16 | 0.84 | 0.98 | 0.67 | 0.37 | 0.19 |
| 3 | 0.17 | 1.07 | 0.79 | 1.01 | 0.71 | 0.39 | 0.19 |
| 4 | 0.31 | 1.09 | 0.88 | 1.07 | 0.70 | 0.36 | 0.17 |
| 5 | 0.30 | 1.34 | 1.16 | 1.21 | 0.88 | 0.58 | 0.26 |
| 6 | 0.33 | 1.01 | 0.74 | 0.87 | 0.66 | 0.39 | 0.14 |
| 7 | 0.29 | 1.26 | 1.03 | 1.26 | 0.79 | 0.49 | 0.22 |
| 8 | 0.31 | 1.26 | 1.05 | 1.27 | 0.85 | 0.46 | 0.23 |

TABLE 2
Adsorbed Methane Content in Respective Subsamples Per
Unit Mass under 30° C. and Different Pressures (m³/t)

| Subsample | Pressure | | | | | | | | | |
|---|---|---|---|---|---|---|---|---|---|---|
| No. | 1 MPa | 2 MPa | 3 MPa | 4 MPa | 5 MPa | 6 MPa | 7 MPa | 8 MPa | 9 MPa | 10 MPa |
| 1 | 1.01 | 1.35 | 1.52 | 1.62 | 1.68 | 1.73 | 1.77 | 1.80 | 1.82 | 1.84 |
| 2 | 0.97 | 1.30 | 1.46 | 1.56 | 1.62 | 1.67 | 1.70 | 1.73 | 1.75 | 1.77 |
| 3 | 1.17 | 1.56 | 1.76 | 1.88 | 1.95 | 2.01 | 2.05 | 2.08 | 2.11 | 2.13 |
| 4 | 1.21 | 1.61 | 1.82 | 1.94 | 2.02 | 2.07 | 2.12 | 2.15 | 2.18 | 2.20 |
| 5 | 1.24 | 1.66 | 1.86 | 1.99 | 2.07 | 2.13 | 2.17 | 2.21 | 2.23 | 2.26 |

2) The mass percentages of organic matter without normalization in 5 subsamples (1.49%, 1.27%, 2.38%, 2.59% and 3.34%) were obtained by substituting the mass percentage of TOC in 5 subsamples (1.28%, 1.10%, 2.07%, 2.22% and 2.94%), the mass percentage of carbon in kerogen (86.12%, 86.72%, 87.01%, 85.57% and 87.98%) into the following equation.

w_{TOM-i}⁰=w_{TOC-i}⁰/w_{C-i}×100%;

where w_{TOM-i}⁰ (%) was an unnormalized mass percentage of organic matter in respective subsamples; w_{TOC-i}⁰ (%) was an experimentally measured mass percentage of organic carbon in respective subsamples; w_{C-i} (%) was an experimentally measured mass percentage of carbon in kerogen in respective subsamples; i was the number of respective subsamples of the mud shale reservoir, and was selected from 1, 2, 3, . . . , n.

Then the mass percentages of organic matter, clay and other minerals in respective subsamples were normalized according to the following equations, where a sum of the mass percentages of organic matter, clay and other minerals in respective subsamples was 100%. The obtained normalized mass percentages of organic matter were respectively 1.49%, 1.27%, 2.38%, 2.59% and 3.34%; the obtained normalized mass percentages of clay were respectively 40.98%, 41.67%, 22.47%, 25.05% and 29.32%; and the obtained normalized mass percentages of other minerals were respectively 57.53%, 57.08%, 75.21%, 72.42% and 67.45% in 5 subsamples.

w_{TOM-i}=w_{TOM-i}⁰×100%

w_{clay-i}=w_{clay-i}⁰×(100−w_{TOM-i}⁰)/100%

wₒₜₕₑᵣₛ₋ᵢ=wₒₜₕₑᵣₛ₋ᵢ⁰×(100−w_{TOM-i}⁰)/100%

where w_{TOM-i} (%), w_{clay-i} (%) and wₒₜₕₑᵣₛ₋ᵢ (%) were normalized mass percentages of organic matter, clay and other minerals in respective subsamples, respectively; w_{TOM-i}⁰, w_{clay-i}⁰ and wₒₜₕₑᵣₛ₋ᵢ⁰ were mass percentages of organic matter, clay and other minerals in respective subsamples before normalization, respectively; i was the number of respective subsamples of the mud shale reservoir, and was selected from 1, 2, 3, . . . , n.

3) A first equation set and a first target function were established according to the normalized mass percentages of organic carbon (1.49%, 1.27%, 2.38%, 2.59% and 3.34%), the normalized mass percentages of clay (40.98%, 41.67%, 22.47%, 25.05% and 29.32%), and the normalized mass percentages of other minerals (57.53%, 57.08%, 75.21%, 72.42% and 67.45%) in respective subsamples obtained in step (2) and the volume Vᵢⱼ (referring to Table 1) of pores having a size respectively of <2 nm, 2-5 nm, 5-10 nm, 10-20 nm, 20-50 nm, 50-100 nm and 100-200 nm in respective sub samples per unit mass obtained in step (1),

where in the case of a minimum value of the first target function f(V_{TOM-j}, V_{clay-j}, Vₒₜₕₑᵣₛ₋ⱼ), a volume of pores having a size numbered as j contributed by organic matter per unit mass was V_{TOM-j} (cm³/g), a volume of pores having a size numbered as j contributed by clay per unit mass was V_{clay-j} (cm³/g), and a volume of pores having a size numbered as j contributed by other minerals per unit mass was Vₒₜₕₑᵣₛ₋ⱼ (cm³/g). The results were shown in Table 3.

$${w}_{TOM \text{-} 1} \times {V}_{TOM \text{-} j} + {w}_{clay \text{-} 1} \times {V}_{clay \text{-} j} + {w}_{others \text{-} 1} \times {V}_{others \text{-} j} = {V}_{1 j}$$

$${w}_{TOM \text{-} 2} \times {V}_{TOM \text{-} j} + {w}_{clay \text{-} 2} \times {V}_{clay \text{-} j} + {w}_{others \text{-} 2} \times {V}_{others \text{-} j} = {V}_{2 j}$$

$${w}_{TOM \text{-} 3} \times {V}_{TOM \text{-} j} + {w}_{clay \text{-} 3} \times {V}_{clay \text{-} j} + {w}_{others \text{-} 3} \times {V}_{others \text{-} j} = {V}_{3 j}$$

$$\begin{matrix}⋯ & ⋯ & ⋯\end{matrix}$$

$${w}_{TOM \text{-} n} \times {V}_{TOM \text{-} j} + {w}_{clay \text{-} n} \times {V}_{clay \text{-} j} + {w}_{others \text{-} n} \times {V}_{others \text{-} j} = {V}_{nj}$$

$${V}_{TOM \text{-} j} > 0 , {V}_{clay \text{-} j} > 0 , {V}_{others \text{-} j} > 0$$

$$f ( {V}_{TOM \text{-} j} , {V}_{clay \text{-} j} , {V}_{others \text{-} j} ) = {\sum }_{i = 1}^{n} {( {V}_{ij} - {w}_{TOM \text{-} i} \times {V}_{TOM \text{-} j} - {w}_{clay \text{-} i} \times {V}_{clay \text{-} j} - {w}_{others \text{-} i} \times {V}_{others \text{-} j} )}^{2}$$

where V_{TOM-j}, V_{clay-j}, Vₒₜₕₑᵣₛ₋ⱼ were the volumes of pores having a size numbered as j respectively contributed by organic matter, clay and other minerals per unit mass; j was a number of pore size from small to large, and was selected from 1, 2, . . . , 7; i was the number of respective subsamples of the mud shale reservoir, and was selected from 1, 2, 3, . . . , 5.

TABLE 3
Volume of Pores Having Different Sizes (×10⁻³ cm³/g)

| | Size | | | | | | |
|---|---|---|---|---|---|---|---|
| Component | <2 nm | 2-5 nm | 5-10 nm | 10-20 nm | 20-50 nm | 50-100 nm | 100-200 nm |
| Organic Matter | 2.2406 | 20.5508 | 19.9608 | 25.1899 | 15.9194 | 10.1544 | 4.7596 |
| Clay | 0.7188 | 2.0910 | 1.3509 | 1.4891 | 1.0590 | 0.5302 | 0.2689 |
| Other Minerals | 0.0111 | 0.0349 | 0.0201 | 0.0498 | 0.0446 | 0.0301 | 0.0263 |

4) A second equation set and a second target function were established using the normalized mass percentages of organic matter (1.49%, 1.27%, 2.38%, 2.59% and 3.34%), the normalized mass percentages of clay (40.98%, 41.67%, 22.47%, 25.05% and 29.32%), and the normalized mass percentages of other minerals (57.53%, 57.08%, 75.21%, 72.42% and 67.45%) in respective subsamples in obtained step (2) and the content Q_{ixy} of adsorbed methane existing in respective subsamples per unit mass (referring to Table 2) obtained in step (1) under a temperature of Tₓ and a pressure of P_{y},

where in the case of a minimum value of the second target function f(Q_{TOM--xy}, Q_{clay-xy}, Q_{others-xy}), a temperature of 30° C. and a pressure respectively of 1 MPa, 2 MPa, 3 MPa, 4 MPa, 5 MPa, 6 MPa, 7 MPa, 8 MPa, 9 MPa and 10 MPa, a content of adsorbed methane existing in organic matter per unit mass was Q_{TOM-xy}, a content of adsorbed methane existing in clay per unit mass was Q_{clay-xy}, and a content of adsorbed methane existing in other minerals per unit mass was Q_{others-xy}. The results were shown in Table 4.

$${w}_{TOM \text{-} 1} \times {Q}_{TOM \text{-} xy} + {w}_{clay \text{-} 1} \times {Q}_{clay \text{-} xy} + {w}_{others \text{-} 1} \times {Q}_{others \text{-} xy} = {Q}_{1 xy}$$

$${w}_{TOM \text{-} 2} \times {Q}_{TOM \text{-} xy} + {w}_{clay \text{-} 2} \times {Q}_{clay \text{-} xy} + {w}_{others \text{-} 2} \times {Q}_{others \text{-} xy} = {Q}_{2 xy}$$

$${w}_{TOM \text{-} 3} \times {Q}_{TOM \text{-} xy} + {w}_{clay \text{-} 3} \times {Q}_{clay \text{-} xy} + {w}_{others \text{-} 3} \times {Q}_{others \text{-} xy} = {Q}_{3 xy}$$

$$\begin{matrix}⋯ & ⋯ & ⋯\end{matrix}$$

$${w}_{TOM \text{-} n} \times {Q}_{TOM \text{-} xy} + {w}_{clay \text{-} n} \times {Q}_{clay \text{-} xy} + {w}_{others \text{-} n} \times {Q}_{others \text{-} xy} = {Q}_{nxy}$$

$${Q}_{TOM \text{-} xy} > 0 , {Q}_{clay \text{-} xy} > 0 , {Q}_{others \text{-} xy} > 0$$

$$f ( {Q}_{TOM \text{-} xy} , {Q}_{clay \text{-} xy} , {Q}_{others \text{-} xy} ) = {\sum }_{i = 1}^{n} {( {Q}_{ixy} - {w}_{TOM \text{-} i} \times {Q}_{TOM \text{-} xy} - {w}_{clay \text{-} i} \times {Q}_{clay \text{-} xy} - {w}_{others \text{-} i} \times {Q}_{others \text{-} xy} )}^{2}$$

where, Q_{TOM-xy} (m³/t), Q_{clay-xy}, (m³/t) and Q_{others-xy} (m³/t) were the contents of adsorbed methane respectively existing in organic matter, clay and other minerals per unit mass under a temperature of Tₓ (° C.) and a pressure P_{y} (MPa);

Q_{ixy} (m³/t) was a content of adsorbed methane existing in subsample i per unit mass under a temperature of Tₓ and a pressure of P_{y}; i was the number of respective subsamples of the mud shale reservoir, and was selected from 1, 2, 3, . . . , 5; x was the number of temperature, and was 1; y was the number of pressure from low to high, and was selected from 1, 2, . . . , 10.

TABLE 4
Content of Adsorbed Methane Existing in Respective Components
Per Unit Mass under Different Pressures (m³/t)

| | Pressure | | | | | | | | | |
|---|---|---|---|---|---|---|---|---|---|---|
| Component | 1 MPa | 2 MPa | 3 MPa | 4 MPa | 5 MPa | 6 MPa | 7 MPa | 8 MPa | 9 MPa | 10 MPa |
| Organic Matter | 27.551 | 40.678 | 47.540 | 51.241 | 53.681 | 55.490 | 56.800 | 57.860 | 58.751 | 59.369 |
| Clay | 1.371 | 2.009 | 2.383 | 2.651 | 2.851 | 2.991 | 3.111 | 3.201 | 3.271 | 3.339 |
| Other Minerals | 0.019 | 0.026 | 0.031 | 0.035 | 0.037 | 0.039 | 0.040 | 0.041 | 0.042 | 0.042 |

5) V_{absorbed by TOM-jxy} was calculated according to the following equations based on step (3) by approximating the pores contributed by organic matter per unit mass to cylinders with corresponding pore size,

where V_{absorbed by TOM-jxy} was a volume of pores occupied by adsorbed methane in pores numbered j contributed by organic matter per unit mass under a temperature of Tₓ and a pressure of P_{y}; when the pores j contributed by organic matter per unit mass had a size D_{TOM-j} lower than 0.38 nm, V_{absorbed by TOM-jxy}=0; when the D_{TOM-j} was not more than twice a thickness h_{absorbed by TOM-jxy of} adsorbed methane and was not less than 0.38 nm, V_{absorbed by TOM-jxy}=V_{TOM-j}; and when the D_{TOM-j} was more than twice the thickness h_{absorbed by TOM-jxy} of adsorbed methane and was not less than 0.38 nm,

$${V}_{absorbed by TOM \text{-} jxy} = \frac{4 {D}_{TOM \text{-} j} \times {h}_{absorbed by TOM \text{-} jxy} - 4 {h}_{absorbed by TOM \text{-} jxy}^{2}}{{D}_{TOM \text{-} j}^{2}} \times {V}_{TOM \text{-} j} . \\ {V}_{absorbed by TOM \text{-} jxy} = \{ \begin{matrix}\begin{matrix}0 & ( {D}_{TOM \text{-} j} < 0.38 nm )\end{matrix} \\ \begin{matrix}{V}_{TOM \text{-} j} & ( 2 {h}_{absorbed by TOM \text{-} jxy} \geq {D}_{TOM \text{-} j} , {D}_{TOM \text{-} j} \geq 0.38 nm )\end{matrix} \\ \frac{4 {D}_{TOM \text{-} j} \times {h}_{absorbed by TOM \text{-} jxy} - 4 {h}_{absorbed by TOM \text{-} jxy}^{2}}{{D}_{TOM \text{-} j}^{2}} \times {V}_{TOM \text{-} j} \\ ( 2 {h}_{absorbed by TOM \text{-} jxy} < {D}_{TOM \text{-} j} , {D}_{TOM \text{-} j} \geq 0.38 nm )\end{matrix}$$

where, V_{absorbed by TOM-jxy} (cm³/g) was the volume of pores occupied by adsorbed methane in pores numbered j contributed by organic matter per unit mass under a temperature of Tₓ and a pressure of P_{y}; V_{TOM-j} (cm³/g) was the volume of pores numbered j contributed by organic matter per unit mass; D_{TOM-j} (nm) was the size of pores numbered j contributed by organic matter; h_{absorbed by TOM-jxy} (nm) was the thickness of adsorbed methane in pores numbered j contributed by organic matter; j was the number of pore sizes from small to large, and was selected from 1, 2, . . . , 7; x was the number of temperature, and was 1; y was the number of pressure from low to high, and was selected from 1, 2, . . . , 10.

V_{absorbed by clay-jxy} was calculated according to the following equations based on step (3) by approximating the pores contributed by clay per unit mass to cylinders with corresponding pore size,

where V_{absorbed by clay-jxy} was a volume of pores occupied by adsorbed methane in pores numbered j contributed by organic matter per unit mass under a temperature of Tₓ and a pressure of P_{y}; when the pores j contributed by organic matter per unit mass had a size D_{clay-j} lower than 0.38 nm, V_{absorbed by clay-jxy}=0; when the D_{clay-j} was not more than twice a thickness h_{absorbed by clay-jxy} of adsorbed methane and was not less than 0.38 nm, V_{absorbed by clay-jxy}=V_{clay-j}; and when the D_{clay-j} was more than twice the thickness h_{absorbed by clay-jxy} of adsorbed methane and was not less than 0.38 nm,

$${V}_{absorbed by clay \text{-} jxy} = \frac{4 {D}_{clay \text{-} j} \times {h}_{absorbed by clay \text{-} jxy} - 4 {h}_{absorbed by clay \text{-} jxy}^{2}}{{D}_{clay \text{-} j}^{2}} \times {V}_{clay \text{-} j} . \\ {V}_{absorbed by clay \text{-} jxy} = \{ \begin{matrix}\begin{matrix}0 & ( {D}_{clay \text{-} j} < 0.38 nm )\end{matrix} \\ \begin{matrix}{V}_{clay \text{-} j} & ( 2 {h}_{absorbed by clay \text{-} jxy} \geq {D}_{clay \text{-} j} , {D}_{clay \text{-} j} \geq 0.38 nm )\end{matrix} \\ \frac{4 {D}_{clay \text{-} j} \times {h}_{absorbed by clay \text{-} jxy} - 4 {h}_{absorbed by clay \text{-} jxy}^{2}}{{D}_{clay \text{-} j}^{2}} \times {V}_{clay \text{-} j} \\ ( 2 {h}_{absorbed by clay \text{-} jxy} < {D}_{clay \text{-} j} , {D}_{clay \text{-} j} \geq 0.38 nm )\end{matrix}$$

where, V_{absorbed by clay-jxy} (cm³/g) was the volume of pores occupied by adsorbed methane in pores numbered j contributed by clay per unit mass under a temperature of Tₓ and a pressure of P_{y}; V_{clay-j} (cm³/g) was the volume of pores numbered j contributed by clay per unit mass; D_{clay-j} (nm) was the size of pores numbered j contributed by clay per unit mass; h_{absorbed by clay-jxy} (nm) was the thickness of adsorbed methane in pores numbered j contributed by clay; j was the number of pore sizes from small to large, and was selected from 1, 2, . . . , 7; x was the number of temperature, and was 1; y was the number of pressure from low to high, and was selected from 1, 2, . . . , 10.

calculating V_{absorbed by others-jxy} according to the following equations based on step (3) by approximating the pores contributed by other minerals per unit mass to cylinders with corresponding pore size;

where V_{absorbed by others-jxy} was a volume of pores occupied by adsorbed methane in pores numbered j contributed by organic matter per unit mass under a temperature of Tₓ and a pressure of P_{y}; when the pores j contributed by organic matter per unit mass had a size Dₒₜₕₑᵣₛ₋ⱼ lower than 0.38 nm, V_{absorbed by others-jxy}=0; when the Dₒₜₕₑᵣₛ₋ⱼ was not more than twice a thickness h_{absorbed by others-jxy} of adsorbed methane and was not less than 0.38 nm, V_{absorbed by others-jxy}=Vₒₜₕₑᵣₛ₋ⱼ; and when the Dₒₜₕₑᵣₛ₋ⱼ was more than twice the thickness h_{absorbed by others-jxy} of adsorbed methane and was not less than 0.38 nm.

$${V}_{absorbed by others \text{-} jxy} = \frac{4 {D}_{others \text{-} j} \times {h}_{absorbed by others \text{-} jxy} - 4 {h}_{absorbed by others \text{-} jxy}^{2}}{{D}_{others \text{-} j}^{2}} \times {V}_{others \text{-} j} . \\ {V}_{absorbed by others \text{-} jxy} = \{ \begin{matrix}\begin{matrix}0 & ( {D}_{others \text{-} j} < 0.38 nm )\end{matrix} \\ \begin{matrix}{V}_{others \text{-} j} & ( 2 {h}_{absorbed by others \text{-} jxy} \geq {D}_{others \text{-} j} , {D}_{others \text{-} j} \geq 0.38 nm )\end{matrix} \\ \frac{4 {D}_{others \text{-} j} \times {h}_{absorbed by others \text{-} jxy} - 4 {h}_{absorbed by others \text{-} jxy}^{2}}{{D}_{others \text{-} j}^{2}} \times {V}_{others \text{-} j} \\ ( 2 {h}_{absorbed by others \text{-} jxy} < {D}_{others \text{-} j} , {D}_{others \text{-} j} \geq 0.38 nm )\end{matrix}$$

where, V_{absorbed by others-jxy} (cm³/g) was the volume of pores occupied by adsorbed methane existing in pores numbered j contributed by other minerals per unit mass under a temperature of Tₓ and a pressure of P_{y}; Vₒₜₕₑᵣₛ₋ⱼ (cm³/g) was the volume of pores numbered j contributed by other minerals per unit mass; Dₒₜₕₑᵣₛ₋ⱼ (nm) was the size of pores numbered j contributed by other minerals per unit mass; h_{absorbed by others-jxy} (nm) was the thickness of adsorbed methane in pores numbered j contributed by other minerals; j was the number of pore sizes from small to large. and was selected from 1, 2, . . . , 7; x was the number of temperature, and was 1; y was the number of pressure from low to high, and was selected from 1, 2, . . . , 10.

6) A third equation set and a third target function were established based on steps (4) and (5) according to the facts that a density of adsorbed methane was lower than that of solid methane but greater than that of free methane; the density of adsorbed methane in pores of organic matter decreased with the increase of pore size; and the density of adsorbed methane decreased with the increase of temperature while increased with the increase of pressure, where in the case of a minimum value of the third target function f(ρ_{absorbed by TOM-jxy}, h_{absorbed by TOM-jxy}), a density ρ_{absorbed by TOM-jxy} and a thickness h_{absorbed by TOM-jxy} of adsorbed methane in pores numbered j contributed by organic matter per unit mass under a temperature of Tₓ and a pressure of P_{y} were obtained. The results were shown in Tables 5 and 6.

$$\frac{22.4}{M} {\sum }_{j = 1}^{7} [ {V}_{absorbed by TOM \text{-} jxy} \times ( {ρ}_{absorbed by TOM \text{-} jxy} - {Σ}_{free \text{-} xy} ) ] = {Q}_{TOM \text{-} xy}$$

$${ρ}_{solid} > {ρ}_{absorbed by TOM \text{-} 1 xy} > {ρ}_{absorbed by TOM \text{-} 2 xy} > … > {ρ}_{absorbed by TOM \text{-} 6 xy} > {ρ}_{absorbed by TOM \text{-} 7 jxy} > {ρ}_{free \text{-} xy}$$

$${ρ}_{solid} > {ρ}_{absorbed by TOM \text{-} j 1 y} > {ρ}_{absorbed by TOM \text{-} j 2 y} > … > {ρ}_{absorbed by TOM \text{-} j ( m - 1 ) y} > {ρ}_{absorbed by TOM \text{-} jmy} > {ρ}_{free \text{-} my}$$

$${ρ}_{solid} > {ρ}_{absorbed by TOM \text{-} jxz} > {ρ}_{absorbed by TOM \text{-} jx ( z - 1 )} > … > {ρ}_{absorbed by TOM \text{-} jx 2} > {ρ}_{absorbed by TOM \text{-} jx 7} > {ρ}_{free \text{-} x 1}$$

$${h}_{absorbed by TOM \text{-} 1 xy} > {h}_{absorbed by TOM \text{-} 2 xy} > … > {h}_{absorbed by TOM \text{-} 6 xy} > {h}_{absorbed by TOM \text{-} 7 xy}$$

$${h}_{absorbed by TOM \text{-} j 1 y} > {h}_{absorbed by TOM \text{-} j 2 y} > … > {h}_{absorbed by TOM \text{-} j ( m - 1 ) y} > {h}_{absorbed by TOM \text{-} jmy}$$

$${h}_{absorbed by TOM \text{-} jxz} > {h}_{absorbed by TOM \text{-} jx ( z - 1 )} > … > {h}_{absorbed by TOM \text{-} jx 2} > {h}_{absorbed by TOM \text{-} jx 1}$$

$$f ( {ρ}_{absorbed by TOM \text{-} jxy} , {h}_{absorbed by TOM \text{-} jxy} ) = {\sum }_{x = 1}^{m} {\sum }_{y = 1}^{z} ( {Q}_{TOM \text{-} xy} - {( \frac{22.4}{M} {\sum }_{j = 1}^{7} [ {V}_{absorbed by TOM \text{-} jxy} \times ( {ρ}_{absorbed by TOM \text{-} jxy} - {ρ}_{free \text{-} xy} ) ] )}^{2}$$

where, V_{absorbed by TOM-jxy} (cm³/g) was the volume of adsorbed methane in pores numbered j contributed by organic matter per unit mass under a temperature of Tₓ and a pressure of P_{y}; ρ_{absorbed by TOM-jxy} (kg/m³) was a density of adsorbed methane in pores numbered j contributed by organic matter per unit mass under a temperature of Tₓ and a pressure of P_{y}; ρ_{free-xy} (kg/m³) was a density of free methane under a temperature of Tₓ and a pressure of P_{y}; Q_{TOM-xy} (m³/t) was the content of adsorbed methane existing in organic matter per unit mass; ρ_{solid} (kg/m³) was a density of solid methane; h_{absorbed by TOM-jxy} (nm) was the thickness of adsorbed methane in pores numbered j contributed by organic matter; M was the molar mass of methane referring to 16.0425 g/mol, j was the number of pore sizes from small to large, and was selected from 1, 2, . . . , 7; x was the number of temperature, and was 1; y was the number of pressure from low to high, and was selected from 1, 2, . . . , 10.

A forth equation set and a forth target function were established based on steps (4) and (5) according to the facts that a density of adsorbed methane was lower than that of solid methane but greater than that of free methane; the density of adsorbed methane in pores of organic matter decreased with the increase of pore size; and the density of adsorbed methane decreased with the increase of temperature while increased with the increase of pressure,

where in the case of a minimum value of the forth target function f(ρ_{absorbed by clay-jxy}, h_{absorbed by clay-jxy}), a density ρ_{absorbed by clay-jxy} and a thickness h_{absorbed by clay-jxy} of adsorbed methane in pores numbered j contributed by clay per unit mass under a temperature of Tₓ and a pressure of P_{y} were obtained. The results were shown in Tables 5 and 6.

$$\frac{22.4}{M} {\sum }_{j = 1}^{7} [ {V}_{absorbed by clay \text{-} jxy} \times ( {ρ}_{absorbed by clay \text{-} jxy} - {Σ}_{free \text{-} xy} ) ] = {Q}_{clay \text{-} xy}$$

$${ρ}_{solid} > {ρ}_{absorbed by clay \text{-} 1 xy} > {ρ}_{absorbed by clay \text{-} 2 xy} > … > {ρ}_{absorbed by clay \text{-} 6 xy} > {ρ}_{absorbed by clay \text{-} 7 jxy} > {ρ}_{free \text{-} xy}$$

$${ρ}_{solid} > {ρ}_{absorbed by clay \text{-} j 1 y} > {ρ}_{absorbed by clay \text{-} j 2 y} > … > {ρ}_{absorbed by clay \text{-} j ( m - 1 ) y} > {ρ}_{absorbed by clay \text{-} jmy} > {ρ}_{free \text{-} my}$$

$${ρ}_{solid} > {ρ}_{absorbed by clay \text{-} jxz} > {ρ}_{absorbed by clay \text{-} jx ( z - 1 )} > … > {ρ}_{absorbed by clay \text{-} jx 2} > {ρ}_{absorbed by clay \text{-} jx 7} > {ρ}_{free \text{-} x 1}$$

$${h}_{absorbed by clay \text{-} 1 xy} > {h}_{absorbed by clay \text{-} 2 xy} > … > {h}_{absorbed by clay \text{-} 6 xy} > {h}_{absorbed by clay \text{-} 7 xy}$$

$${h}_{absorbed by clay \text{-} j 1 y} > {h}_{absorbed by clay \text{-} j 2 y} > … > {h}_{absorbed by clay \text{-} j ( m - 1 ) y} > {h}_{absorbed by clay \text{-} jmy}$$

$${h}_{absorbed by clay \text{-} jxz} > {h}_{absorbed by clay \text{-} jx ( z - 1 )} > … > {h}_{absorbed by clay \text{-} jx 2} > {h}_{absorbed by clay \text{-} jx 1}$$

$$f ( {ρ}_{absorbed by clay \text{-} jxy} , {h}_{absorbed by clay \text{-} jxy} ) = {\sum }_{x = 1}^{m} {\sum }_{y = 1}^{z} ( {Q}_{clay \text{-} xy} - {( \frac{22.4}{M} {\sum }_{j = 1}^{7} [ {V}_{absorbed by clay \text{-} jxy} \times ( {ρ}_{absorbed by clay \text{-} jxy} - {ρ}_{free \text{-} xy} ) ] )}^{2}$$

where, V_{absorbed by clay-jxy} (cm³/g) was the volume of adsorbed methane in pores numbered j contributed by clay per unit mass under a temperature of Tₓ and a pressure of P_{y}; ρ_{absorbed by clay-jxy} (kg/m³) was a density of adsorbed methane in pores numbered j contributed by clay per unit mass under a temperature of Tₓ and a pressure of P_{y}; ρ_{free-xy} (kg/m³) was the density of free methane under a temperature of Tₓ and a pressure of P_{y}; Q_{clay-xy} (m³/t) was the content of adsorbed methane existing in clay per unit mass; ρ_{solid} (kg/m³) was the density of solid methane; h_{absorbed by clay-jxy} (nm) was the thickness of adsorbed methane in pores numbered j contributed by clay; M was the molar mass of methane referring to 16.0425 g/mol; j was the number of pore sizes from small to large, and was selected from 1, 2, . . . , 7; x was the number of temperature, and was 1; y was the number of pressure from low to high, and was selected from 1, 2,. . . , 10.

A fifth equation set and a fifth target function were established based on steps (4) and (5) according to the facts that a density of adsorbed methane was lower than that of solid methane but greater than that of free methane; the density of adsorbed methane in pores of organic matter decreased with the increase of pore size; and the density of adsorbed methane decreased with the increase of temperature while increased with the increase of pressure,

where in the case of a minimum value of the fifth target function f(ρ_{absorbed by others-jxy}, h_{absorbed by others-jxy}), a density ρ_{absorbed by others-jxy} and a thickness h_{absorbed by others-jxy} of adsorbed methane in pores numbered j contributed by other minerals per unit mass under a temperature of Tₓ and a pressure of P_{y} were obtained. The results were shown in Tables 5 and 6.

$$\frac{22.4}{M} {\sum }_{j = 1}^{7} [ {V}_{absorbed by others \text{-} jxy} \times ( {ρ}_{absorbed by others \text{-} jxy} - {Σ}_{free \text{-} xy} ) ] = {Q}_{others \text{-} xy}$$

$${ρ}_{solid} > {ρ}_{absorbed by others \text{-} 1 xy} > {ρ}_{absorbed by others \text{-} 2 xy} > … > {ρ}_{absorbed by others \text{-} 6 xy} > {ρ}_{absorbed by others \text{-} 7 jxy} > {ρ}_{free \text{-} xy}$$

$${ρ}_{solid} > {ρ}_{absorbed by others \text{-} j 1 y} > {ρ}_{absorbed by others \text{-} j 2 y} > … > {ρ}_{absorbed by others \text{-} j ( m - 1 ) y} > {ρ}_{absorbed by others \text{-} jmy} > {ρ}_{free \text{-} my}$$

$${ρ}_{solid} > {ρ}_{absorbed by others \text{-} jxz} > {ρ}_{absorbed by others \text{-} jx ( z - 1 )} > … > {ρ}_{absorbed by others \text{-} jx 2} > {ρ}_{absorbed by others \text{-} jx 7} > {ρ}_{free \text{-} x 1}$$

$${h}_{absorbed by others \text{-} 1 xy} > {h}_{absorbed by others \text{-} 2 xy} > … > {h}_{absorbed by others \text{-} 6 xy} > {h}_{absorbed by others \text{-} 7 xy}$$

$${h}_{absorbed by others \text{-} j 1 y} > {h}_{absorbed by others \text{-} j 2 y} > … > {h}_{absorbed by others \text{-} j ( m - 1 ) y} > {h}_{absorbed by others \text{-} jmy}$$

$${h}_{absorbed by others \text{-} jxz} > {h}_{absorbed by others \text{-} jx ( z - 1 )} > … > {h}_{absorbed by others \text{-} jx 2} > {h}_{absorbed by others \text{-} jx 1}$$

$$f ( {ρ}_{absorbed by others \text{-} jxy} , {h}_{absorbed by others \text{-} jxy} ) = {\sum }_{x = 1}^{m} {\sum }_{y = 1}^{z} ( {Q}_{others \text{-} xy} - {( \frac{22.4}{M} {\sum }_{j = 1}^{7} [ {V}_{absorbed by others \text{-} jxy} \times ( {ρ}_{absorbed by others \text{-} jxy} - {ρ}_{free \text{-} xy} ) ] )}^{2}$$

where, V_{absorbed by others-jxy} (cm³/g) was the volume of adsorbed methane in pores numbered j contributed by other minerals per unit mass under a temperature of Tₓ and a pressure of P_{y}; ρ_{absorbed by others-jxy} (kg/m³) was a density of adsorbed methane in pores numbered j contributed by other minerals per unit mass under a temperature of Tₓ and a pressure of P_{y}; ρ_{free-xy} (kg/m³) was the density of free methane under a temperature of Tₓ and a pressure of P_{y}; Q_{others-xy} (m³/t) was the content of adsorbed methane existing in other minerals per unit mass; ρ_{solid} (kg/m³) was the density of solid methane; h_{absorbed by others-jxy} (nm) was the thickness of adsorbed methane in pores numbered j contributed by other minerals; M was the molar mass of methane referring to 16.0425 g/mol; j was the number of pore sizes from small to large, and was selected from 1, 2, . . . , 7; x was the number of temperature, and was 1; y was the number of pressure from low to high, and was selected from 1, 2, . . . , 10.

TABLE 5

| | Pore Size | Density of Adsorbed methane in pores Having Respective Sizes under Respective Pressures (kg/m³) | | | | | | | | | |
|---|---|---|---|---|---|---|---|---|---|---|---|
| Component | (nm) | 1 MPa | 2 MPa | 3 MPa | 4 MPa | 5 MPa | 6 MPa | 7 MPa | 8 MPa | 9 MPa | 10 MPa |
| Organic | <2 | 452.63 | 635.60 | 728.12 | 775.30 | 807.20 | 829.22 | 845.39 | 860.38 | 870.66 | 877.92 |
| Matter | 2-5 | 448.96 | 630.78 | 719.34 | 766.99 | 796.69 | 819.32 | 836.45 | 848.95 | 859.02 | 866.83 |
| | 5-10 | 437.83 | 614.82 | 702.90 | 747.29 | 777.49 | 800.38 | 814.97 | 828.00 | 839.12 | 846.94 |
| | 10-20 | 427.05 | 600.75 | 686.84 | 732.39 | 762.73 | 783.29 | 798.22 | 810.83 | 822.41 | 828.32 |
| | 20-50 | 410.78 | 575.47 | 658.32 | 703.69 | 730.91 | 751.57 | 765.50 | 778.57 | 788.49 | 794.31 |
| | 50-100 | 380.07 | 547.00 | 629.61 | 670.91 | 699.30 | 720.39 | 735.90 | 745.45 | 756.52 | 763.12 |
| | 100-200 | 342.16 | 500.95 | 579.57 | 619.99 | 646.01 | 668.69 | 680.11 | 693.11 | 700.59 | 705.99 |
| Clay | <2 | 277.93 | 380.88 | 440.13 | 482.09 | 512.09 | 534.39 | 553.86 | 567.65 | 576.70 | 586.50 |
| | 2-5 | 270.89 | 373.34 | 431.75 | 473.40 | 503.83 | 525.73 | 542.79 | 556.11 | 566.85 | 577.30 |
| | 5-10 | 255.88 | 357.60 | 413.67 | 453.99 | 485.46 | 505.60 | 525.11 | 538.56 | 548.33 | 557.90 |
| | 10-20 | 249.12 | 349.88 | 405.40 | 444.22 | 474.94 | 494.61 | 511.75 | 524.88 | 535.73 | 544.80 |
| | 20-50 | 232.33 | 327.89 | 383.58 | 423.24 | 451.78 | 469.60 | 485.81 | 499.27 | 508.66 | 518.30 |
| | 50-100 | 207.36 | 297.09 | 347.99 | 385.80 | 411.57 | 429.38 | 444.90 | 456.88 | 465.46 | 475.70 |
| | 100-200 | 176.93 | 257.54 | 301.87 | 335.31 | 359.71 | 376.37 | 389.71 | 400.26 | 407.46 | 415.30 |
| Other | <2 | 239.25 | 297.12 | 338.46 | 374.39 | 388.60 | 406.21 | 412.40 | 419.29 | 424.37 | 426.96 |
| Minerals | 2-5 | 231.30 | 288.36 | 329.11 | 364.77 | 379.09 | 396.41 | 404.22 | 410.97 | 415.65 | 418.87 |
| | 5-10 | 220.37 | 274.93 | 313.20 | 344.79 | 361.00 | 376.21 | 384.16 | 388.81 | 393.71 | 395.03 |
| | 10-20 | 206.76 | 260.47 | 300.77 | 331.99 | 347.17 | 363.21 | 369.61 | 374.87 | 379.40 | 382.33 |
| | 20-50 | 185.63 | 240.64 | 277.59 | 308.89 | 324.40 | 339.81 | 346.22 | 352.89 | 355.61 | 359.36 |
| | 50-100 | 167.33 | 214.30 | 247.05 | 276.18 | 288.13 | 302.61 | 308.32 | 312.40 | 317.47 | 319.89 |
| | 100-200 | 146.19 | 186.98 | 213.98 | 237.88 | 247.59 | 258.61 | 263.20 | 268.65 | 272.70 | 274.03 |

TABLE 6

| | Pore Size | Thickness of Adsorbed methane in pores Having Respective Sizes under Respective Pressures (nm) | | | | | | | | | |
|---|---|---|---|---|---|---|---|---|---|---|---|
| Component | (nm) | 1 MPa | 2 MPa | 3 MPa | 4 MPa | 5 MPa | 6 MPa | 7 MPa | 8 MPa | 9 MPa | 10 MPa |
| Organic | <2 | 1.47 | 1.62 | 1.70 | 1.74 | 1.76 | 1.78 | 1.80 | 1.81 | 1.82 | 1.82 |
| Matter | 2-5 | 1.43 | 1.59 | 1.67 | 1.71 | 1.74 | 1.76 | 1.77 | 1.78 | 1.79 | 1.80 |
| | 5-10 | 1.35 | 1.52 | 1.60 | 1.64 | 1.67 | 1.69 | 1.71 | 1.72 | 1.73 | 1.73 |
| | 10-20 | 1.27 | 1.44 | 1.52 | 1.56 | 1.59 | 1.61 | 1.63 | 1.64 | 1.65 | 1.66 |
| | 20-50 | 1.08 | 1.23 | 1.31 | 1.35 | 1.37 | 1.39 | 1.41 | 1.42 | 1.43 | 1.43 |
| | 50-100 | 0.84 | 0.97 | 1.04 | 1.07 | 1.09 | 1.11 | 1.12 | 1.13 | 1.14 | 1.14 |
| | 100-200 | 0.52 | 0.66 | 0.73 | 0.76 | 0.78 | 0.80 | 0.81 | 0.82 | 0.83 | 0.84 |
| Clay | <2 | 0.99 | 1.13 | 1.22 | 1.28 | 1.32 | 1.35 | 1.38 | 1.40 | 1.41 | 1.43 |
| | 2-5 | 0.95 | 1.10 | 1.18 | 1.24 | 1.28 | 1.31 | 1.34 | 1.36 | 1.37 | 1.39 |
| | 5-10 | 0.90 | 1.05 | 1.13 | 1.18 | 1.23 | 1.26 | 1.28 | 1.30 | 1.32 | 1.33 |
| | 10-20 | 0.84 | 0.97 | 1.05 | 1.10 | 1.14 | 1.17 | 1.19 | 1.21 | 1.22 | 1.24 |
| | 20-50 | 0.71 | 0.83 | 0.90 | 0.95 | 0.98 | 1.01 | 1.03 | 1.04 | 1.06 | 1.07 |
| | 50-100 | 0.51 | 0.63 | 0.69 | 0.74 | 0.78 | 0.80 | 0.82 | 0.84 | 0.85 | 0.86 |
| | 100-200 | 0.31 | 0.40 | 0.45 | 0.49 | 0.52 | 0.54 | 0.56 | 0.57 | 0.58 | 0.59 |
| Other | <2 | 0.86 | 0.96 | 1.03 | 1.09 | 1.12 | 1.16 | 1.16 | 1.17 | 1.18 | 1.19 |
| Minerals | 2-5 | 0.84 | 0.93 | 1.00 | 1.06 | 1.09 | 1.13 | 1.13 | 1.14 | 1.15 | 1.16 |
| | 5-10 | 0.78 | 0.87 | 0.94 | 1.00 | 1.03 | 1.06 | 1.07 | 1.08 | 1.09 | 1.09 |
| | 10-20 | 0.70 | 0.80 | 0.88 | 0.94 | 0.97 | 1.00 | 1.01 | 1.02 | 1.03 | 1.04 |
| | 20-50 | 0.58 | 0.66 | 0.73 | 0.78 | 0.80 | 0.83 | 0.84 | 0.85 | 0.85 | 0.86 |
| | 50-100 | 0.39 | 0.47 | 0.53 | 0.59 | 0.61 | 0.64 | 0.65 | 0.66 | 0.66 | 0.67 |
| | 100-200 | 0.24 | 0.30 | 0.34 | 0.38 | 0.39 | 0.42 | 0.42 | 0.42 | 0.43 | 0.43 |

1. A method for evaluating thickness and density of adsorbed methane in pores contributed by organic matter, clay and other minerals in a mud shale reservoir, comprising:
1) crushing a mud shale reservoir sample to produce a plurality of subsamples; and selecting three or more subsamples varying in mesh for determinations of organic carbon content and kerogen content, whole rock analysis, and determinations of low temperature nitrogen adsorption-desorption and methane isotherm adsorption;
wherein:
mass percentages of organic carbon in respective subsamples are w_{TOC-1}⁰, w_{TOC-2}⁰, . . . and w_{TOC-n}⁰ (%), respectively; mass percentages of carbon in kerogen in respective subsamples are w_{C-1}, w_{C-2}, and w_{C-n} (%), respectively; mass percentages of clay in respective subsamples are w_{clay-1}⁰, w_{clay-2}⁰, . . . and w_{clay-n}⁰ (%), respectively; and mass percentages of other minerals in respective subsamples are wₒₜₕₑᵣₛ₋₁⁰, wₒₜₕₑᵣₛ₋₂⁰, . . . and wₒₜₕₑᵣₛ₋ₙ⁰ (%), respectively, pores in respective subsamples per unit mass having a size respectively of <2 nm, 2-5 nm, 5-10 nm, 10-20 nm, 20-50 nm, 50-100 nm and 100-200 nm have a volume of Vᵢⱼ (cm³/ g); respective subsamples per unit mass have an adsorbed methane content of Q_{ixy} (m³/t) under a temperature of Tₓ and a pressure of P_{y},
wherein i is the number of respective subsamples of the mud shale reservoir, and is selected from 1, 2, 3, . . . , n; j is the number of pore sizes, selected from 1, 2, . . . , 7; x is the number of temperature from low to high, and is selected from 1, 2, . . . , m; y is the number of pressure from low to high, and is selected from 1, 2, . . . , z;
2) substituting the mass percentages of organic carbon (w_{TOC-1}⁰, w_{TOC-2}⁰, . . . and w_{TOC-n}⁰) and the corresponding mass percentages of carbon in kerogen (w_{C-1}, w_{C-2}, . . . and w_{C-n}) in respective subsamples into the following equation to obtain mass percentages of organic matter in respective subsamples (w_{TOM-1}⁰, w_{TOM-2}⁰, . . . and w_{TOM-n}⁰);
w_{TOM-i}⁰=w_{TOM-i}⁰/w_{C-i}×100%;
wherein w_{TOM-i}⁰ (%) is an unnormalized mass percentage of organic matter in respective subsamples; w_{TOC-i}⁰ (%) is an experimentally measured mass percentage of organic carbon in respective subsamples; w_{C-i} (%) is an experimentally measured mass percentage of carbon in kerogen in respective subsamples; i is the number of respective subsamples of the mud shale reservoir, and is selected from 1, 2, 3, . . . , n; and
normalizing the mass percentages of organic matter, clay and other minerals in respective subsamples according to the following equations; wherein a sum of the mass percentages of organic matter, clay and other minerals in respective subsamples is 100%; the normalized mass percentages of organic matter, clay and other minerals is in respective subsamples are respectively w_{TOM-i} (%), w_{clay-i} (%) and wₒₜₕₑᵣₛ₋ᵢ (%);
w_{TOM-i}=w_{TOM-i}⁰×100%
w_{clay-i}=w_{clay-i}⁰×(100−w_{TOM-i}⁰)/100%
wₒₜₕₑᵣₛ₋ᵢ=wₒₜₕₑᵣₛ₋ᵢ⁰×(100−w_{TOM-i}⁰)/100%
wherein w_{TOM-i}⁰, w_{clay-i}⁰ and wₒₜₕₑᵣₛ₋ᵢ⁰ are mass percentages of organic matter, clay and other minerals in respective subsamples before normalization, respectively; i is the number of respective subsamples of the mud shale reservoir, and is selected from 1, 2, 3, . . . , n;
3) establishing a first equation set and a first target function according to the normalized mass percentages of organic matter (w_{TOM-1}, w_{TOM-2}, . . . and w_{TOM-n}), the normalized mass percentages of clay (w_{clay-1}, w_{clay-2}, . . . and w_{clay-n}), and the normalized mass percentage of other minerals (wₒₜₕₑᵣₛ₋₁, wₒₜₕₑᵣₛ₋₂, . . . and wₒₜₕₑᵣₛ₋ₙ) in respective subsamples obtained in step (2) and the volume Vᵢⱼ (cm³/g) of pores having a size respectively of <2 nm, 2-5 nm, 5-10 nm, 10-20 nm, 20-50 nm, 50-100 nm and 100-200 nm in respective subsamples per unit mass obtained in step (1);
wherein in the case of a minimum value of the first target function f(V_{TOM-j}, V_{clay-j}, Vₒₜₕₑᵣₛ₋ⱼ), a volume of pores having a size numbered as j contributed by organic matter per unit mass is V_{TOM-j} (cm³/g), a volume of pores having a size numbered as j contributed by clay per unit mass is V_{clay-j}, (cm³/g), and a volume of pores having a size numbered as j contributed by other minerals per unit mass is Vₒₜₕₑᵣₛ₋ⱼ (cm³/g);
$${w}_{TOM \text{-} 1} \times {V}_{TOM \text{-} j} + {w}_{clay \text{-} 1} \times {V}_{clay \text{-} j} + {w}_{others \text{-} 1} \times {V}_{others \text{-} j} = {V}_{1 j}$$
$${w}_{TOM \text{-} 2} \times {V}_{TOM \text{-} j} + {w}_{clay \text{-} 2} \times {V}_{clay \text{-} j} + {w}_{others \text{-} 2} \times {V}_{others \text{-} j} = {V}_{2 j}$$
$${w}_{TOM \text{-} 3} \times {V}_{TOM \text{-} j} + {w}_{clay \text{-} 3} \times {V}_{clay \text{-} j} + {w}_{others \text{-} 3} \times {V}_{others \text{-} j} = {V}_{13}$$
$$… … …$$
$${w}_{TOM \text{-} n} \times {V}_{TOM \text{-} j} + {w}_{clay \text{-} n} \times {V}_{clay \text{-} j} + {w}_{others \text{-} n} \times {V}_{others \text{-} j} = {V}_{nj}$$
$${V}_{TOM \text{-} j} > 0 , {V}_{clay \text{-} j} > 0 , {V}_{others \text{-} j} > 0$$
$$f ( {V}_{TOM \text{-} j} , {V}_{clay \text{-} j} , {V}_{others \text{-} j} ) = {\sum }_{i = 1}^{n} {( {V}_{ij} - {w}_{TOM \text{-} i} \times {V}_{TOM \text{-} i} - {w}_{clay \text{-} i} \times {V}_{clay \text{-} j} - {w}_{others \text{-} i} \times {V}_{others \text{-} j} )}^{2} ;$$
wherein V_{TOM-j}, V_{clay-j} and Vₒₜₕₑᵣₛ₋ⱼ are the volumes of pores having a size numbered as j respectively contributed by organic matter, clay and other minerals per unit mass; j is a number of pore size from small to large, and is selected from 1, 2, . . . 6 and 7; i is the number of respective subsamples of the mud shale reservoir, and is selected from 1, 2, 3, . . . , n;
4) establishing a second equation set and a second target function using the normalized mass percentage of organic matter (w_{TOM-1}, w_{TOM-2}, . . . and w_{TOM-n}), the normalized mass percentage of clay (w_{clay-1}, w_{clay-2}, . . . and w_{clay-n}), and the normalized mass percentage of other minerals (wₒₜₕₑᵣₛ₋₋₁, wₒₜₕₑᵣₛ₋₂, . . . and wₒₜₕₑᵣₛ₋ₙ) in respective subsamples obtained in step (2) and the content Q_{ixy} of adsorbed methane existing in respective subsamples per unit mass obtained in step (1) under a temperature of Tₓ and a pressure of P_{y};
wherein in the case of a minimum value of the second target function f(Q_{TOM-xy}, Q_{clay-xy}, Q_{others-xy}), a temperature of Tₓ and a pressure of P_{y}, a content of adsorbed methane existing in organic matter per unit mass is Q_{TOM-xy}, a content of adsorbed methane existing in clay per unit mass is Q_{clay-xy}, and a content of adsorbed methane existing in other minerals per unit mass is Q_{others-xy};
$${w}_{TOM \text{-} 1} \times {Q}_{TOM \text{-} xy} + {w}_{clay \text{-} 1} \times {Q}_{clay \text{-} xy} + {w}_{others \text{-} 1} \times {Q}_{others \text{-} xy} = {Q}_{1 xy}$$
$${w}_{TOM \text{-} 2} \times {Q}_{TOM \text{-} xy} + {w}_{clay \text{-} 2} \times {Q}_{clay \text{-} xy} + {w}_{others \text{-} 2} \times {Q}_{others \text{-} xy} = {Q}_{2 xy}$$
$${w}_{TOM \text{-} 3} \times {Q}_{TOM \text{-} xy} + {w}_{clay \text{-} 3} \times {Q}_{clay \text{-} xy} + {w}_{others \text{-} 3} \times {Q}_{others \text{-} xy} = {Q}_{3 xy}$$
$$… … …$$
$${w}_{TOM \text{-} n} \times {Q}_{TOM \text{-} xy} + {w}_{clay \text{-} n} \times {Q}_{clay \text{-} xy} + {w}_{others \text{-} n} \times {Q}_{others \text{-} xy} = {Q}_{nxy}$$
$${Q}_{TOM \text{-} xy} > 0 , {Q}_{clay \text{-} xy} > 0 , {Q}_{others \text{-} xy} > 0$$
$$f ( {Q}_{TOM \text{-} xy} , {Q}_{clay \text{-} xy} , {Q}_{others \text{-} xy} ) = {\sum }_{i = 1}^{n} {( {Q}_{ixy} - {w}_{TOM \text{-} i} \times {Q}_{TOM \text{-} xy} - {w}_{clay \text{-} i} \times {Q}_{clay \text{-} xy} - {w}_{others \text{-} i} \times {Q}_{others \text{-} xy} )}^{2}$$
wherein, Q_{TOM-xy} (m³/t), Q_{clay-xy} (m³/t) and Q_{others-xy} (m³/t) are the contents of adsorbed methane respectively existing in organic matter, clay and other minerals per unit mass under a temperature of Tₓ (° C.) and a pressure P_{y} (MPa);
Q_{ixy} (m³/t) is a content of adsorbed methane existing in subsample i per unit mass under a temperature of Tₓ and a pressure of P_{y}; wherein i is the number of respective subsamples of the mud shale reservoir, and is selected from 1, 2, 3, . . . , n; x is the number of temperature from low to high, and is selected from 1, 2, . . . , m; and y is the number of pressure from low to high, and is selected from 1, 2, . . . , z;
5) calculating V_{absorbed by TOM-jxy} according to the following equations based on step (3) by approximating the pores contributed by organic matter per unit mass to cylinders with corresponding pore size;
wherein V_{absorbed by TOM-jxy} is the volume of pores occupied by adsorbed methane in pores numbered j contributed by organic matter per unit mass under a temperature of Tₓ and a pressure of P_{y}; when the pores j contributed by organic matter per unit mass have a size D_{TOM-j} lower than 0.38 nm, V_{absorbed by TOM-jxy}=0; when the D_{TOM-j} is not more than twice a thickness h_{absorbed by TOM-jxy} of adsorbed methane and is not less than 0.38 nm, V_{absorbed by TOM-jxy}=V_{TOM-j}; and when the D_{TOM-j} is more than twice the thickness h_{absorbed by TOM-jxy} of adsorbed methane and is not less than 0.38 nm,
$${V}_{absorbed by TOM \text{-} jxy} = \frac{4 {D}_{TOM \text{-} j} \times {h}_{absorbed by TOM \text{-} jxy} - 4 {h}_{absorbed by TOM \text{-} jxy}^{2}}{{D}_{TOM \text{-} j}^{2}} \times {V}_{TOM \text{-} j} ;$$
$${V}_{absorbed by TOM \text{-} jxy} = \{ \begin{matrix}0 & ( {D}_{TOM \text{-} j} < 0.38 nm ) \\ {V}_{TOM \text{-} j} & ( 2 {h}_{absorbed by TOM \text{-} jxy} \geq {D}_{TOM \text{-} j} , {D}_{TOM \text{-} j} \geq 0.38 nm ) \\ \frac{4 {D}_{TOM \text{-} j} \times {h}_{absorbed by TOM \text{-} jxy} - 4 {h}_{absorbed by TOM \text{-} jxy}^{2}}{{D}_{TOM \text{-} j}^{2}} \times {V}_{TOM \text{-} j} & ( 2 {h}_{absorbed by TOM \text{-} jxy} < {D}_{TOM \text{-} j} , {D}_{TOM \text{-} j} \geq 0.38 nm )\end{matrix} ;$$
wherein, V_{absorbed by TOM-jxy} (cm³/g) is the volume of pores occupied by adsorbed methane in pores numbered j contributed by organic matter per unit mass under a temperature of Tₓ and a pressure of P_{y}; V_{TOM-j} (cm³/g) is the volume of pores numbered j contributed by organic matter per unit mass, D_{TOM-j} (nm) is the size of pores numbered j contributed by organic matter; h_{absorbed by TOM-jxy} (nm) is the thickness of adsorbed methane in pores numbered j contributed by organic matter; j is the number of pore sizes from small to large, and is selected from 1, 2, . . . , 7; x is the number of temperature from low to high, and is selected from 1, 2, . . . , m; and y is the number of pressure from low to high, and is selected from 1, 2, . . . , z;
calculating V_{absorbed by clay-jxy} according to the following equations based on step (3) by approximating the pores contributed by clay per unit mass to cylinders with corresponding pore size;
wherein V_{absorbed by clay-jxy} is the volume of pores occupied by adsorbed methane in pores numbered j contributed by organic matter per unit mass under a temperature of Tₓ and a pressure of P_{y}; when the pores j contributed by organic matter per unit mass have a size D_{clay-j} lower than 0.38 nm, V_{absorbed by clay-jxy}=0; when the D_{clay-j} is not more than twice a thickness h_{absorbed by clay-jxy} of adsorbed methane and is not less than 0.38 nm, V_{absorbed by clay-jxy}=V_{clay-j}; and when the D_{clay-j} is more than twice the thickness h_{absorbed by clay-jxy} of adsorbed methane and is not less than 0.38 nm.
$${V}_{absorbed by clay \text{-} jxy} = \frac{4 {D}_{clay \text{-} j} \times {h}_{absorbed by clay \text{-} jxy} - 4 {h}_{absorbed by clay \text{-} jxy}^{2}}{{D}_{clay \text{-} j}^{2}} \times {V}_{clay \text{-} j} ;$$
$${V}_{absorbed by clay \text{-} jxy} = \{ \begin{matrix}0 & ( {D}_{clay \text{-} j} < 0.38 nm ) \\ {V}_{clay \text{-} j} & ( 2 {h}_{absorbed by clay \text{-} jxy} \geq {D}_{clay \text{-} j} , {D}_{clay \text{-} j} \geq 0.38 nm ) \\ \frac{4 {D}_{clay \text{-} j} \times {h}_{absorbed by clay \text{-} jxy} - 4 {h}_{absorbed by clay \text{-} jxy}^{2}}{{D}_{clay \text{-} j}^{2}} \times {V}_{clay \text{-} j} & ( 2 {h}_{absorbed by clay \text{-} jxy} < {D}_{clay \text{-} j} , {D}_{clay \text{-} j} \geq 0.38 nm )\end{matrix} ;$$
wherein, V_{absorbed by clay-jxy} (cm³/g) is the volume of pores occupied by adsorbed methane in pores numbered j contributed by clay per unit mass under a temperature of Tₓ and a pressure of P_{y}; V_{clay-j} (cm³/g) is the volume of pores numbered j contributed by clay per unit mass; D_{clay-j} (nm) is the size of pores numbered j contributed by clay per unit mass; h_{absorbed by clay-jxy} (nm) is the thickness of adsorbed methane in pores numbered j contributed by clay; j is the number of pore sizes from small to large, and is selected from 1, 2, . . . , 7, x is the number of temperature from low to high, and is selected from 1, 2, . . . , m; and y is the number of pressure from low to high, and is selected from 1, 2, . . . , z;
calculating V_{absorbed by others-jxy} according to the following equations based on step (3) by approximating the pores contributed by other minerals per unit mass to cylinders with corresponding pore size;
wherein V_{absorbed by others-jxy} is the volume of pores occupied by adsorbed methane in pores numbered j contributed by organic matter per unit mass under a temperature of Tₓ and a pressure of P_{y}; when the pores j contributed by organic matter per unit mass have a size Dₒₜₕₑᵣₛ₋ⱼ lower than 0.38 nm, V_{absorbed by others-jxy}=0; when the Dₒₜₕₑᵣₛ₋ⱼ is not more than twice a thickness h_{absorbed by others-jxy} of adsorbed methane and is not less than 0.38 nm, V_{absorbed by others-jxy}=Vₒₜₕₑᵣₛ₋ⱼ; and when the Dₒₜₕₑᵣₛ₋ⱼ is more than twice the thickness h_{absorbed by others-jxy} of adsorbed methane and is not less than 0.38 nm,
$${V}_{absorbed by others \text{-} jxy} = \frac{4 {D}_{others \text{-} j} \times {h}_{absorbed by others \text{-} jxy} - 4 {h}_{absorbed by others \text{-} jxy}^{2}}{{D}_{others \text{-} j}^{2}} \times {V}_{others \text{-} j} ;$$
$${V}_{absorbed by others \text{-} jxy} = \{ \begin{matrix}0 & ( {D}_{others \text{-} j} < 0.38 nm ) \\ {V}_{others \text{-} j} & ( 2 {h}_{absorbed by others \text{-} jxy} \geq {D}_{others \text{-} j} , {D}_{others \text{-} j} \geq 0.38 nm ) \\ \frac{4 {D}_{others \text{-} j} \times {h}_{absorbed by others \text{-} jxy} - 4 {h}_{absorbed by others \text{-} jxy}^{2}}{{D}_{others \text{-} j}^{2}} \times {V}_{others \text{-} j} & ( 2 {h}_{absorbed by others \text{-} jxy} < {D}_{others \text{-} j} , {D}_{others \text{-} j} \geq 0.38 nm )\end{matrix} ;$$
wherein , V_{absorbed by others-jxy} (cm³/g) is the volume of pores occupied by adsorbed methane existing in pores numbered j contributed by other minerals per unit mass under a temperature of Tₓ and a pressure of P_{y}; Vₒₜₕₑᵣₛ₋ⱼ (cm³/g) is the volume of pores numbered j contributed by other minerals per unit mass; Dₒₜₕₑᵣₛ₋ⱼ (nm) is the size of pores numbered j contributed by other minerals per unit mass; h_{absorbed by others-jxy} (nm) is the thickness of adsorbed methane in pores numbered j contributed by other minerals; j is the number of pore sizes from small to large, and is selected from 1, 2, . . . , 7; x is the number of temperature from low to high, and is selected from 1, 2, . . . , m; and y is the number of pressure from low to high, and is selected from 1, 2, . . . , z;
6) establishing a third equation set and a third target function based on steps (4) and (5) according to the facts that a density of adsorbed methane is lower than that of solid methane but greater than that of free methane; the density of adsorbed methane in pores of organic matter decreases with the increase of pore size, and the density of adsorbed methane decreases with the increase of temperature while increases with the increase of pressure;
wherein in the case of a minimum value of the third target function f(ρ_{absorbed by TOM-jxy}, h_{absorbed by TOM-jxy}), a density ρ_{absorbed by TOM-jxy} and a thickness h_{absorbed by TOM-jxy} of adsorbed methane in pores numbered j contributed by organic matter per unit mass under a temperature of Tₓ and a pressure of P_{y} can be obtained;
$$\frac{22.4}{M} {\sum }_{j = 1}^{7} [ {V}_{absorbed by TOM \text{-} jxy} \times ( {ρ}_{absorbed by TOM \text{-} jxy} - {Σ}_{free \text{-} xy} ) ] = {Q}_{TOM \text{-} xy}$$
$${ρ}_{solid} > {ρ}_{absorbed by TOM \text{-} 1 xy} > {ρ}_{absorbed by TOM \text{-} 2 xy} > … > {ρ}_{absorbed by TOM \text{-} 6 xy} > {ρ}_{absorbed by TOM \text{-} 7 jxy} > {ρ}_{free \text{-} xy}$$
$${ρ}_{solid} > {ρ}_{absorbed by TOM \text{-} j 1 y} > {ρ}_{absorbed by TOM \text{-} j 2 y} > … > {ρ}_{absorbed by TOM \text{-} j ( m - 1 ) y} > {ρ}_{absorbed by TOM \text{-} jmy} > {ρ}_{free \text{-} my}$$
$${ρ}_{solid} > {ρ}_{absorbed by TOM \text{-} jxz} > {ρ}_{absorbed by TOM \text{-} jx ( z - 1 )} > … > {ρ}_{absorbed by TOM \text{-} jx 2} > {ρ}_{absorbed by TOM \text{-} jx 7} > {ρ}_{free \text{-} x 1}$$
$${h}_{absorbed by TOM \text{-} 1 xy} > {h}_{absorbed by TOM \text{-} 2 xy} > … > {h}_{absorbed by TOM \text{-} 6 xy} > {h}_{absorbed by TOM \text{-} 7 xy}$$
$${h}_{absorbed by TOM \text{-} j 1 y} > {h}_{absorbed by TOM \text{-} j 2 y} > … > {h}_{absorbed by TOM \text{-} j ( m - 1 ) y} > {h}_{absorbed by TOM \text{-} jmy}$$
$${h}_{absorbed by TOM \text{-} jxz} > {h}_{absorbed by TOM \text{-} jx ( z - 1 )} > … > {h}_{absorbed by TOM \text{-} jx 2} > {h}_{absorbed by TOM \text{-} jx 1}$$
$$f ( {ρ}_{absorbed by TOM \text{-} jxy} , {h}_{absorbed by TOM \text{-} jxy} ) = {\sum }_{x = 1}^{m} {\sum }_{y = 1}^{z} {( {Q}_{TOM \text{-} xy} - \frac{22.4}{M} {\sum }_{j = 1}^{7} [ {V}_{absorbed by TOM \text{-} jxy} \times ( {ρ}_{absorbed by TOM \text{-} jxy} - {ρ}_{free \text{-} xy} ) ] )}^{2} ;$$
wherein, V_{absorbed by TOM-jxy} (cm³/g) is the volume of adsorbed methane in pores numbered j contributed by organic matter per unit mass under a temperature of Tₓ and a pressure of P_{y}; ρ_{absorbed by TOM-jxy} (kg/m³) is a density of adsorbed methane in pores numbered j contributed by organic matter per unit mass under a temperature of Tₓ and a pressure of P_{y}; ρ_{free-xy} (kg/m³) is a density of free methane under a temperature of Tₓ and a pressure of P_{y}; Q_{TOM-xy} (m³/t) is the content of adsorbed methane existing in organic matter per unit mass; ρ_{solid} (kg/m³) is a density of solid methane; h_{absorbed by TOM-jxy} (nm) is the thickness of adsorbed methane in pores numbered j contributed by organic matter; M is the molar mass of methane referring to 16.0425 g/mol; j is the number of pore sizes from small to large, and is selected from 1, 2, . . . , 7; x is the number of temperature from low to high, and is selected from 1, 2, . . . , m; and y is the number of pressure from low to high, and is selected from 1, 2, . . . , z;
establishing a forth equation set and a forth target function based on steps (4) and (5) according to the facts that the density of adsorbed methane is lower than that of solid methane but greater than that of free methane, the density of adsorbed methane in the pores of clay decreases with the increase of pore size, and the density of adsorbed methane decreases with the increase of temperature while increases with the increase of pressure;
wherein in the case of a minimum value of the forth target function f(ρ_{absorbed by clay-jxy}, h_{absorbed by clay-jxy}), a density ρ_{absorbed by clay-jxy} and a thickness h_{absorbed by clay-jxy} of adsorbed methane in pores numbered j contributed by clay per unit mass under a temperature of Tₓ and a pressure of P_{y} can be obtained;
$$\frac{22.4}{M} {\sum }_{j = 1}^{7} [ {V}_{absorbed by clay \text{-} jxy} \times ( {ρ}_{absorbed by clay \text{-} jxy} - {Σ}_{free \text{-} xy} ) ] = {Q}_{clay \text{-} xy}$$
$${ρ}_{solid} > {ρ}_{absorbed by clay \text{-} 1 xy} > {ρ}_{absorbed by clay \text{-} 2 xy} > … > {ρ}_{absorbed by clay \text{-} 6 xy} > {ρ}_{absorbed by clay \text{-} 7 jxy} > {ρ}_{free \text{-} xy}$$
$${ρ}_{solid} > {ρ}_{absorbed by clay \text{-} j 1 y} > {ρ}_{absorbed by clay \text{-} j 2 y} > … > {ρ}_{absorbed by clay \text{-} j ( m - 1 ) y} > {ρ}_{absorbed by clay \text{-} jmy} > {ρ}_{free \text{-} my}$$
$${ρ}_{solid} > {ρ}_{absorbed by clay \text{-} jxz} > {ρ}_{absorbed by clay \text{-} jx ( z - 1 )} > … > {ρ}_{absorbed by clay \text{-} jx 2} > {ρ}_{absorbed by clay \text{-} jx 7} > {ρ}_{free \text{-} x 1}$$
$${h}_{absorbed by clay \text{-} 1 xy} > {h}_{absorbed by clay \text{-} 2 xy} > … > {h}_{absorbed by clay \text{-} 6 xy} > {h}_{absorbed by clay \text{-} 7 xy}$$
$${h}_{absorbed by clay \text{-} j 1 y} > {h}_{absorbed by clay \text{-} j 2 y} > … > {h}_{absorbed by clay \text{-} j ( m - 1 ) y} > {h}_{absorbed by clay \text{-} jmy}$$
$${h}_{absorbed by clay \text{-} jxz} > {h}_{absorbed by clay \text{-} jx ( z - 1 )} > … > {h}_{absorbed by clay \text{-} jx 2} > {h}_{absorbed by clay \text{-} jx 1}$$
$$f ( {ρ}_{absorbed by clay \text{-} jxy} , {h}_{absorbed by clay \text{-} jxy} ) = {\sum }_{x = 1}^{m} {\sum }_{y = 1}^{z} {( {Q}_{clay \text{-} xy} - \frac{22.4}{M} {\sum }_{j = 1}^{7} [ {V}_{absorbed by clay \text{-} jxy} \times ( {ρ}_{absorbed by clay \text{-} jxy} - {ρ}_{free \text{-} xy} ) ] )}^{2} ;$$
wherein, V_{absorbed by clay-jxy} (cm³/g) is the volume of adsorbed methane in pores numbered j contributed by clay per unit mass under a temperature of Tₓ and a pressure of P_{y}; ρ_{absorbed by clay-jxy} (kg/m³) is a density of adsorbed methane in pores numbered j contributed by clay per unit mass under a temperature of Tₓ and a pressure of P_{y}; ρ_{free-xy} (kg/m³) is the density of free methane under a temperature of Tₓ and a pressure of P_{y}; Q_{clay-xy} (m³/t) is the content of adsorbed methane existing in clay per unit mass; ρ_{solid} (kg/m³) is the density of solid methane; h_{absorbed by clay-jxy} (nm) is the thickness of adsorbed methane in pores numbered j contributed by clay; M is the molar mass of methane referring to 16.0425 g/mol; j is the number of pore sizes from small to large, and is selected from 1, 2, . . . , 7; x is the number of temperature from low to high, and is selected from 1, 2, . . . , m; y is the number of pressure from low to high, and is selected from 1, 2, . . . , z;
establishing a fifth equation set and a fifth target function based on steps (4) and (5) according to the facts that the density of adsorbed methane is lower than that of solid methane but greater than that of free methane, the density of adsorbed methane in the pores of other minerals decreases with the increase of pore size, and the density of adsorbed methane decreases with the increase of temperature while increases with the increase of pressure;
wherein in the case of a minimum value of the fifth target function f(ρ_{absorbed by others-jxy}, h_{absorbed by others-jxy}), a density ρ_{absorbed by others-jxy} and a thickness h_{absorbed by others-jxy} of adsorbed methane in pores numbered j contributed by other minerals per unit mass under a temperature of Tₓ and a pressure of P_{y} can be obtained;
$$\frac{22.4}{M} {\sum }_{j = 1}^{7} [ {V}_{absorbed by others \text{-} jxy} \times ( {ρ}_{absorbed by others \text{-} jxy} - {Σ}_{free \text{-} xy} ) ] = {Q}_{others \text{-} xy}$$
$${ρ}_{solid} > {ρ}_{absorbed by others \text{-} 1 xy} > {ρ}_{absorbed by others \text{-} 2 xy} > … > {ρ}_{absorbed by others \text{-} 6 xy} > {ρ}_{absorbed by others \text{-} 7 jxy} > {ρ}_{free \text{-} xy}$$
$${ρ}_{solid} > {ρ}_{absorbed by others \text{-} j 1 y} > {ρ}_{absorbed by others \text{-} j 2 y} > … > {ρ}_{absorbed by others \text{-} j ( m - 1 ) y} > {ρ}_{absorbed by others \text{-} jmy} > {ρ}_{free \text{-} my}$$
$${ρ}_{solid} > {ρ}_{absorbed by others \text{-} jxz} > {ρ}_{absorbed by others \text{-} jx ( z - 1 )} > … > {ρ}_{absorbed by others \text{-} jx 2} > {ρ}_{absorbed by others \text{-} jx 7} > {ρ}_{free \text{-} x 1}$$
$${h}_{absorbed by others \text{-} 1 xy} > {h}_{absorbed by others \text{-} 2 xy} > … > {h}_{absorbed by others \text{-} 6 xy} > {h}_{absorbed by others \text{-} 7 xy}$$
$${h}_{absorbed by others \text{-} j 1 y} > {h}_{absorbed by others \text{-} j 2 y} > … > {h}_{absorbed by others \text{-} j ( m - 1 ) y} > {h}_{absorbed by others \text{-} jmy}$$
$${h}_{absorbed by others \text{-} jxz} > {h}_{absorbed by others \text{-} jx ( z - 1 )} > … > {h}_{absorbed by others \text{-} jx 2} > {h}_{absorbed by others \text{-} jx 1}$$
$$f ( {ρ}_{absorbed by others \text{-} jxy} , {h}_{absorbed by others \text{-} jxy} ) = {\sum }_{x = 1}^{m} {\sum }_{y = 1}^{z} {( {Q}_{others \text{-} xy} - \frac{22.4}{M} {\sum }_{j = 1}^{7} [ {V}_{absorbed by others \text{-} jxy} \times ( {ρ}_{absorbed by others \text{-} jxy} - {ρ}_{free \text{-} xy} ) ] )}^{2} ;$$
wherein, V_{absorbed by others-jxy} (cm³/g) is the volume of adsorbed methane in pores numbered j contributed by other minerals per unit mass under a temperature of Tₓ and a pressure of P_{y}; ρ_{absorbed by others-jxy} (kg/m³) is a density of adsorbed methane in pores numbered j contributed by other minerals per unit mass under a temperature of Tₓ and a pressure of P_{y}; ρ_{free-xy} (kg/m³) is the density of free methane under a temperature of Tₓ and a pressure of P_{y}; Q_{others-xy} (m³/t) is the content of adsorbed methane existing in other minerals per unit mass; ρ_{solid} (kg/m³) is the density of solid methane; h_{absorbed by others-jxy} (nm) is the thickness of adsorbed methane in pores numbered j contributed by other minerals; M is the molar mass of methane referring to 16.0425 g/mol; j is the number of pore sizes from small to large, and is selected from 1, 2, . . . , 7; x is the number of temperature from low to high, and is selected from 1, 2, . . . , m; y is the number of pressure from low to high, and is selected from 1, 2, . . . , z.
